(19) United States
(12) Patent Application Publication
(10) Pub. No.: US 2022/0264877 A1
(43) Pub. Date: Aug. 25, 2022

(54) MICROBIOCIDAL PICOLINAMIDE DERIVATIVES

(71) Applicant: SYNGENTA CROP PROTECTION AG, Basel (CH)

(72) Inventors: Mathias BLUM, Stein (CH); Clemens LAMBERTH, Stein (CH); Renaud BEAUDEGNIES, Stein (CH); Martin POULIOT, Stein (CH); Stefano RENDINE, Stein (CH)

(73) Assignee: SYNGENTA CROP PROTECTION AG, Basel (CH)

(21) Appl. No.: 17/624,641

(22) PCT Filed: Jul. 3, 2020
(86) PCT No.: PCT/EP2020/068893

§ 371 Date: Jan. 4, 2022

(30) Foreign Application Priority Data
Jul. 5, 2019 (EP) 19184781.3

(51) Int. Cl. A01N 43/40; C07D 213/81; A01P 7/04

(52) U.S. Cl. CPC A01N 43/40; A01P 7/04; C07D 213/81

(57) ABSTRACT

Compounds of the formula (I) wherein the substituents are as defined in claim 1, useful as pesticides, and especially fungicides.

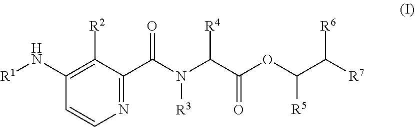

The present invention relates to microbiocidal picolinamide derivatives, e.g., as active ingredients, which have microbiocidal activity, in particular fungicidal activity. The invention also relates to the preparation of these picolinamide derivatives, to agrochemical compositions which comprise at least one of the picolinamide derivatives and to uses of the picolinamide derivatives or compositions thereof in agriculture or horticulture for controlling or preventing the infestation of plants, harvested food crops, seeds or non-living materials by phytopathogenic microorganisms, preferably fungi.

Picolinamide compounds as fungicidal agents are described in WO 2016/109257, WO 2016/109288, WO 2016/109289, WO 2016/109300, WO 2016/109301, WO 2016/109302, WO 2016/109303, WO 2019/068809, WO 2019/068812 and WO2019/121149.

According to the present invention, there is provided a compound of formula (I):

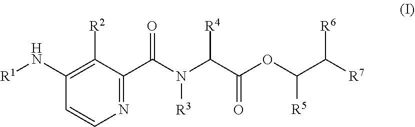

wherein,

R¹ is hydrogen, formyl, C₁-C₁₂alkylcarbonyl, C₁-C₆alkoxycarbonyl, C₁-C₆haloalkylcarbonyl, C₁-C₆alkoxyC1-C₆alkylcarbonyl, or C₁-C₆haloalkoxycarbonyl;

R² is hydroxyl, C₂-C₆acyloxy, C₂-C₆haloacyloxy, C₁-C₆alkoxyC1-C₆alkoxy, C₁-C₆haloalkoxyC₁-C₆alkoxy, C₁-C₆alkoxyC₁-C₆haloalkoxy, C₁-C₆acyloxyC₁-C₆alkoxy, C₂-C₆haloacyloxyC₁-C₆alkoxy, or C₂-C₆acyloxyC₁-C₆haloalkoxy;

R³ is hydrogen, C₁-C₆alkyl, C₁-C₆alkoxy or C₃-C₈cycloalkyl;

R⁴ and R⁵ are each independently C₁-C₁₂alkyl, C₃-C₈cycloalkyl, C₁-C₆haloalkyl, or C₁-C₆alkoxyC₁-C₆alkyl, wherein each C₃-C₈cycloalkyl moiety is optionally substituted with 1, 2 or 3 halogen atoms which may be the same or different;

R⁶ is C₁-C₁₂alkyl, C₃-C₈cycloalkyl, phenyl or heteroaryl, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸. or

R⁵ and R⁶ together with the carbon atoms to which they are attached may form a 3-, 4-, 5- or 6-membered cycloalkyl or heterocyclyl ring, wherein the heterocyclic moiety is a stable 3-, 4-, 5- or 6-membered non-aromatic monocyclic ring which comprises 1, 2 or 3 heteroatoms, wherein the heteroatoms are individually selected from N, O and S;

R⁷ is phenyl, phenoxy, heteroaryl or heteroaryloxy, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸;

R⁸ is hydroxyl, halogen, cyano, C₁₋C₆alkyl, C₂₋C₆alkenyl, C₂₋C₆alkynyl, C₁₋C₄haloalkyl, cyanoC₁₋C₆alkyl, hydroxyC₁₋C₆alkyl, or C₁₋C₄alkoxyC₁₋C₆alkyl;

or a salt or an N-oxide thereof.

Surprisingly, it has been found that the novel compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I) according to the present invention. Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I), or a composition comprising this compound as active ingredient, is applied to the plants, to parts thereof or the locus thereof.

According to a fourth aspect of the invention, there is provided the use of a compound of formula (I) as a fungicide. According to this particular aspect of the invention, the use may exclude methods for the treatment of the human or animal body by surgery or therapy.

Where substituents are indicated as being “optionally substituted”, this means that they may or may not carry one or more identical or different substituents, e.g., one, two or three R⁸ substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not be limited to, CH₂ClO—, CHCl₂O—, CCl₃O—, CH₂FO—, CHF₂O—, CF₃O—, CF₃CH₂O— or CH₃CF₂O— groups.

As used herein, the term “hydroxy” or“hydroxy” means a —OH group.

As used herein, the term “cyano” means a —CN group.

As used herein, the term “halogen” refers to fluorine (fluoro), chlorine (chloro), bromine (bromo) or iodine (iodo).

As used herein, the term “formyl” means a —C(═O)H group.

As used herein, the term “C₁-C₆alky” refers to a straight or branched hydrocarbon chain radical consisting solely of carbon and hydrogen atoms, containing no unsaturation, having from one to six carbon atoms, and which is attached to the rest of the molecule by a single bond. The terms “C₁-C₁₂alky” and “C₁-C₄alkyl” are to be construed accordingly. Examples of C₁-C₆alkyl include, but are not limited to, methyl, ethyl, n-propyl, n-butyl, n-pentyl, n-hexyl and the isomers thereof, for example, iso-propyl, iso-butyl, sec-butyl, tert-butyl or iso-amyl. A “C₁-C₆alkylene” group refers to the corresponding definition of C₁-C₆alkyl, except that such radical is attached to the rest of the molecule by two single bonds. The term “C₁₋C₂alkylene” is to be construed accordingly. Examples of C₁-C₆alkylene, include, but are not limited to, —CH₂—, —CH₂CH₂— and —(CH₂)₃—.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. Examples of C₂-C₆alkenyl include, but are not limited to, ethenyl (vinyl), prop-1-enyl, prop-2-enyl (allyl), and but-1-enyl.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. Examples of C₂-C₆alkynyl include, but are not limited to, ethynyl, prop-1-ynyl, and but-1-ynyl.

As used herein, the term “C₂-C₆cycloalkyl” refers to a radical which is a monocyclic saturated ring system and which contains 3 to 8 carbon atoms. The term “C₃-C₆cycloalkyl” is to be construed accordingly. Examples of C₃-C₈cycloalkyl include, but are not limited to, cyclopropyl, 1-methylcyclopropyl, 2-methylcyclopropyl, cyclobutyl, cyclopentyl, cyclohexyl, cycloheptyl and cyclooctyl.

As used herein, the term “C₁-C₆alkoxy” refers to a radical of the formula —ORₐ where Rₐ is a C₁-C₆alkyl radical as generally defined above. The term “C₁-C₄alkoxy” is to be construed accordingly. Examples of C₁-C₆alkoxy include, but are not limited to, methoxy, ethoxy, 1-methylethoxy (iso-propoxy), propoxy, butoxy, 1-methylpropoxy and 2-methylpropoxy.

As used herein, the term “C₁-C₆alkoxyC1-C₆alkoxy” refers to a radical of the formula R_{b}O—RₐO— where R_{b} is a C₁-C₆alkyl radical as generally defined above, and Rₐ is a C₁-C₆alkyl radical as generally defined above. Examples of C₁-C₆alkoxyC₁-C₆alkoxy include, but are not limited to, methoxymethoxy, ethoxymethoxy and methoxyethoxy.

As used herein, the term “C₁-C₆haloalkoxyC₁-C₆alkoxy” refers to a radical of the formula R_{b}O—RₐO—, where Rₐ is a C₁-C₆alkyl radical as generally defined above and R_{b} is a C₁-C₆alkyl radical as generally defined above substituted by one or more of the same or different halogen atoms. Examples of C₁-C₆haloalkoxyC₁-C₆alkoxy groups include, but not limited to trifluoromethoxymethoxy.

As used herein, the term “C₁-C₆alkoxyC₁-C₆haloalkoxy” refers to a radical of the formula RₐO—R_{b}O—, where Rₐ is a C₁-C₆alkyl radical as generally defined above and R_{b} is a C₁-C₆alkyl radical as generally defined above substituted by one or more of the same or different halogen atoms. Examples of C₁-C₆alkoxyC₁-C₆haloalkoxy groups include, but not limited to methoxydifluoromethoxy.

As used herein, the term “C₂-C₆acyl” refers to a radical RₐC(═O)—, where Rₐ is a C₁-C₅alkyl or a C₃-C₅cycloalkyl radical as generally defined above. Acyl groups include, but are not limited to, acetyl, propanoyl and cyclopropanoyl.

As used herein, the term “C₂-C₆acyloxy” refers to a radical of the formula —OR_{b} where R_{b} is a C₂-C₆acyl, or a C₃-C₅cycloalkyl radical as generally defined above. C₂-C₆acyloxy groups include, but are not limited to, acetoxy, propanoyloxy, isopropanoyloxy, butanoyloxy, and cyclopropanoyloxy.

As used herein, the term “C₂-C₆haloacyloxy” refers to a radical of the formula RₐC(═O)O—, where Rₐ is C₁-C₅alkyl or a C₃-C₅cycloalkyl radical as generally defined above substituted by one or more of the same or different halogen atoms. C₂-C₆haloacyloxy groups include, but are not limited to trifluoroacetoxy.

As used herein, the term “C₂-C₆acyloxyC₁-C₆alkoxy” refers to a radical of the formula RₐC(═O)OR_{b}O— where Rₐ is a C₁-C₅alkyl or a C₃-C₅cycloalkyl radical as generally defined above and where R_{b} is a C₁-C₆alkyl radical as generally defined above.

As used herein, the term “C₂-C₆haloacyloxyC₁-C₆alkoxy” refers to a radical of the formula RₐC(═O)OR_{b}O— where Rₐ is a C₁-C₅alkyl or a C₃-C₅cycloalkyl radical as generally defined above substituted by one or more of the same or different halogen atoms, and R_{b} is a C₁-C₆alkyl radical as generally defined above.

As used herein, the term “C₂-C₆acyloxyC₁-C₆haloalkoxy” refers to a radical of the formula RₐC(═O)OR_{b}O— where Rₐ is a C₁-C₅alkyl or a C₃-C₅cycloalkyl radical as generally defined above, and R_{b} is a C₁-C₆alkyl radical as generally defined above substituted by one or more of the same or different halogen atoms.

As used herein, the term “C₁-C₆alkoxyC₁-C₆haloacyloxy” refers to a radical of the formula RₐOR_{b}C(═O)O— where Rₐ is a C₁-C₆alkyl radical as generally defined above, and R_{b} is a C₁-C₅alkyl radical as generally defined above substituted by one or more of the same or different halogen atoms.

As used herein, the term “cyanoC₁-C₆alkyl” refers to a C₁-C₆alkylene radical as generally defined above substituted by one or more cyano groups as defined above.

As used herein, the term “hydroxyC₁-C₆alkyl” refers to a C₁-C₆alkylene radical as generally defined above substituted by one or more hydroxyl groups as defined above.

As used herein, the term “C₁-C₆alkylcarbonyl” refers to a radical of the formula —C(O)Rₐ where Rₐ is a C₁₋₆alkyl radical as generally defined above. The terms “C₁-C₁₂alkylcabonyl” and “C₁-C₄alkylcarbonyl” are to be construed accordingly. Examples of C₁-C₆alkylcarbonyl include, but are not limited to, methylcarbonyl, ethylcarbonyl, n-propylcarbonyl, n-butylcarbonyl, n-pentylcarbonyl, n-hexylcarbonyl and the isomers thereof, for example, iso-propylcarbonyl, iso-butylcarbonyl, sec-butylcarbonyl, tert-butylcarbonyl or iso-amylcarbonyl.

As used herein, the term “C₁-C₆alkoxycarbonyl” refers to a radical of the formula —C(O)ORₐ where Rₐ is a C₁-C₆alkyl radical as generally defined above. The term “C₁-C₄alkoxycarbonyl” is to be construed accordingly. Examples of C₁-C₆alkoxycarbonyl include, but are not limited to, methoxycarbonyl, ethoxycarbonyl, 1-methylethoxycarbonyl (iso-propoxycarbonyl), propoxycarbonyl, butoxycarbonyl 1-methylpropoxycarbonyl and 2-methylpropoxycarbonyl.

As used herein, the term “C₁-C₆haloalkylcarbonyl” refers to a radical of the formula —C(O)Rₐ where Rₐ is a C₁-C₆alkyl radical as generally defined above substituted by one or more of the same or different halogen atoms. Examples of C₁-C₆haloalkylcarbonyl include, but are not limited to trifluoromethylcarbonyl.

As used herein, the term “C₁-C₆alkoxyC₁-C₆alkylcarbonyl” refers to a radical of the formula —C(O)R_{b}ORₐ where R_{b} is a C₁-C₆alkyl radical as generally defined above, and Rₐ is a C₁-C₆alkyl radical as generally defined above. Examples of C₁-C₆alkoxyC₁-C₆alkylcarbonyl include, but are not limited to, methoxymethylcarbonyl.

As used herein, the term “C₁-C₆haloalkoxycarbonyl” refers to a radical of the formula —C(O)ORₐ where Rₐ is a C₁-C₆alkyl radical as generally defined above substituted by one or more of the same or different halogen atoms. Examples of C₁-C₆haloalkoxycarbonyl groups include, but are not limited to trifluoromethoxycarbonyl.

As used herein, the term “heterocyclyl” or “heterocyclic” refers to a stable 3-, 4-, 5- or 6-membered non-aromatic monocyclic ring which comprises 1, 2 or 3 heteroatoms, wherein the heteroatoms are individually selected from N, O and S. The heterocyclyl radical may be bonded to the rest of the molecule via a carbon atom or heteroatom. Examples of heterocyclyl include, but are not limited to, aziridinyl, azetidinyl, oxetanyl, thietanyl, tetrahydrofuryl, pyrrolidinyl, pyrazolidinyl, imidazolidnyl, piperidinyl, piperazinyl, morpholinyl, dioxolanyl, dithiolanyl and thiazolidinyl.

As used herein, the term “heteroaryl” refers to a 5- or 6-membered aromatic monocyclic ring radical which comprises 1, 2, 3 or 4 heteroatoms individually selected from N, O and S. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

As used herein, the term “heteroaryloxy” refers to a radical of the formula —ORₐ where Rₐ is a heteroaryl radical as generally defined above. Examples of heteroaryloxy include, but are not limited to, pyridyloxy and thienyloxy.

As used herein, ═O means an oxo group, e.g., as found in a carbonyl (—C(═O)—) group.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, in oxidized form as an N-oxide, or in salt form, e.g., an agronomically usable salt form.

N-oxides are oxidized forms of tertiary amines or oxidized forms of nitrogen-containing heteroaromatic compounds. They are described for instance in the book “Heterocyclic N-oxides” by A. Albini and S. Pietra, CRC Press, Boca Raton (1991).

The following list provides definitions, including preferred definitions, for substituents R¹, R², R³, R⁴, R⁵, R⁶, R⁷ and R⁸, with reference to compounds of formula (I). For any one of these substituents, any of the definitions given below may be combined with any definition of any other substituent given below or elsewhere in this document.

R¹ is hydrogen, formyl, C₁-C₁₂alkylcarbonyl, C₁-C₆alkoxycarbonyl, C₁-C₆haloalkylcarbonyl, C₁-C₆alkoxyC₁-C₆alkylcarbonyl, or C₁-C₆haloalkoxycarbonyl. Preferably, R¹ is hydrogen, formyl, C₁-C₆alkylcarbonyl, C₁-C₆alkoxycarbonyl, or C₁-C₆alkoxyC₁-C₆alkylcarbonyl, more preferably, hydrogen, formyl, C₁-C₆alkylcarbonyl, or C₁-C₆alkoxycarbonyl, even more preferably hydrogen, formyl, C₁-C₄alkylcarbonyl, or C₁-C₄alkoxycarbonyl. More preferably still, R¹ is hydrogen, formyl or C₁-C₄alkylcarbonyl, and most preferably R¹ is hydrogen or formyl.

R² is hydroxyl, C₂-C₆acyloxy, C₂-C₆haloacyloxy, C₁-C₆alkoxyC₁-C₆alkoxy, C₁-C₆haloalkoxyC₁-C₆alkoxy, C₁-C₆alkoxyC₁-C₆haloalkoxy, C₂-C₆acyloxyC₁-C₆alkoxy, C₂-C₆haloacyloxyC₁-C₆alkoxy, or C₂-C₆acyloxyC₁-C₆haloalkoxy. Preferably, R² is hydroxyl, C₂-C₆acyloxy, C₁-C₆alkoxyC₁-C₆alkoxy or C₂-C₆acyloxyC₁-C₆alkoxy, more preferably hydroxyl, C₂-C₆acyloxy or C₂-C₆acyloxyC₁-C₆alkoxy, and even more preferably hydroxyl, acetoxy or isobutyryloxymethoxy. Most preferably, R² is hydroxyl or isobutyryloxymethoxy.

R³ is hydrogen, C₁-C₆alkyl, C₁-C₆alkoxy or C₃-C₈cycloalkyl. Preferably R³ is hydrogen, C₁-C₆alkyl, C₁-C₆alkoxy or C₃-C₆cycloalkyl. More preferably R³ is, hydrogen, C₁-C₄alkyl, C₁-C₄alkoxy or C₃-C₆cycloalkyl. Even more preferably R³ is, hydrogen, methoxy or cyclopropyl, more preferably still hydrogen or methoxy, and most preferably, R³ is hydrogen.

R⁴ and R⁵ are each independently C₁-C₁₂alkyl, C₃-C₈cycloalkyl, C₁-C₆haloalkyl, C₁-C₆alkoxyC₁-C₆alkyl, wherein each C₃-C₆cycloalkyl moiety is optionally substituted with 1, 2 or 3 halogen atoms which may be the same or different. Preferably, R⁴ and R⁵ are each independently C₁-C₆alkyl, C₃-C₆cycloalkyl, C₁-C₆haloalkyl, or C₁-C₆alkoxyC₁-C₆alkyl. More preferably, R⁴ and R⁵ are each independently C₁-C₆alkyl, C₃-C₆cycloalkyl or C₁-C₆haloalkyl, even more preferably C₁-C₄alkyl, C₃-C₆cycloalkyl or C₁-C₄haloalkyl, more preferably still, C₁-C₄alkyl or C₁-C₄haloalkyl, even more preferably still, C₁-C₄alkyl, and most preferably R⁴ and R⁵ are each independently methyl or ethyl. In one set of embodiments, R⁴ and R⁵ are both methyl.

Rₐ is C₁-C₁₂alkyl, C₃-C₈cycloalkyl, phenyl or heteroaryl, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸. Preferably, R⁶ is C₁-C₆alkyl, C₃-C₆cycloalkyl, phenyl or heteroaryl, wherein the heteroaryl moiety is a 5- or 6-membered aromatic ring which comprises 1, 2 or 3 heteroatoms individually selected from N, O and S, and wherein the phenyl and heteroaryl moieties are optionally substituted by 1, 2 or 3 substituents, which may be the same or different, selected from R⁸.

More preferably, R⁶ is C₁-C₆alkyl, phenyl or heteroaryl, wherein the heteroaryl moiety is a 5- or 6-membered aromatic ring which comprises 1 or 2 heteroatoms individually selected from N, O and S (e.g. pyridyl), and wherein the phenyl and heteroaryl moieties are optionally substituted by 1 or 2 substituents, which may be the same or different, selected from R⁸. More preferably still, R is C₁-C₆alkyl or phenyl, wherein the phenyl moiety is optionally substituted by 1 or 2 substituents, which may be the same or different, selected from R⁸. Even more preferably, R⁶ is isopropyl, phenyl or 4-fluorophenyl. Most preferably, R⁶ is phenyl, or 4-fluorophenyl; or

R⁵ and R⁶ together with the carbon atoms to which they are attached may form a 3-, 4-, 5- or 6-membered cycloalkyl or heterocycloalkyl ring, wherein the heterocyclic moiety is a stable 3-, 4-, 5- or 6-membered non-aromatic monocyclic ring which comprises 1, 2 or 3 heteroatoms, wherein the heteroatoms are individually selected from N, O and S, preferably a 3-, 4-, 5- or 6-membered cycloalkyl ring, and more preferably cyclopropyl, cyclopentyl or cyclohexyl.

R⁷ is phenyl, phenoxy, heteroaryl or heteroaryloxy, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸. Preferably, R⁷ is phenyl, phenoxy, heteroaryl or heteroaryloxy, wherein the heteroaryl moiety is a 5- or 6-membered aromatic ring which comprises 1, 2 or 3 heteroatoms individually selected from N, O and S, and wherein the phenyl and heteroaryl moieties are optionally substituted by 1, 2 or 3 substituents, which may be the same or different, selected from R⁸. More preferably, R⁷ is phenyl, phenoxy or heteroaryl, wherein the heteroaryl moiety is a 5- or 6-membered aromatic ring which comprises 1 or 2 nitrogen atoms (e.g. pyridyl), and wherein the phenyl and heteroaryl moieties are optionally substituted by 1 or 2 substituents, which may be the same or different, selected from R⁸. Even more preferably, R⁷ is phenyl or phenoxy, wherein the phenyl moieties are optionally substituted by one R⁸. More preferably still, R⁷ is phenyl, 4-fluorophenyl, phenoxy or 4-fluorophenoxy. Most preferably, R⁷ is phenyl, or 4-fluorophenyl.

In one set of embodiments, R⁶ and R⁷ are both phenyl. In another set of embodiments, R⁶ and R⁷ are both 4-fluorophenyl.

R⁸ is hydroxyl, halogen, cyano, C₁-C₆alkyl, C₃-C₆alkenyl, C₃-C₆alkynyl, C₁₋C₄haloalkyl, cyanoC₁-C₆alkyl, hydroxyC₁-C₆alkyl, or C₁-C₄alkoxyC₁-C₆alkyl. Preferably, R⁸ is hydroxyl, halogen, cyano, C₁-C₆alkyl or C₁₋C₄haloalkyl, more preferably, hydroxyl, chloro, fluoro, methyl, cyano, difluoromethyl, or trifluoromethyl, even more preferably, chloro and fluoro, and most preferably, R⁸ is fluoro.

In a compound of formula (I) according to the present invention, preferably:
R¹ is hydrogen, formyl, C₁-C₁₂alkylcarbonyl, C₁-C₆alkoxycarbonyl, C₁-C₆haloalkylcarbonyl, C₁-C₆alkoxyC₁-C₆alkylcarbonyl, or C₁-C₆haloalkoxycarbonyl;
R² is hydroxyl, C₂-C₆acyloxy or C₂-C₆acyloxyC₁-C₆alkoxy;
R³ is hydrogen;
R⁴ is methyl;
R⁵ is methyl;
R⁶ is C₁-C₁₂alkyl, C₃-C₈cycloalkyl, phenyl or heteroaryl, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from Rₐ;
R⁷ is phenyl, phenoxy, heteroaryl or heteroaryloxy, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from Rₐ; and
R⁸ is hydroxyl, halogen, cyano, C₁₋₆alkyl, or C₁₋₄haloalkyl.

More preferably, R¹ is hydrogen, formyl, C₁-C₁₂alkylcarbonyl, C₁-C₆alkoxycarbonyl, C₁-C₆haloalkylcarbonyl, C₁-C₆alkoxyC₁-C₆alkylcarbonyl, or C₁-C₆haloalkoxycarbonyl;
R² is hydroxyl, C₂-C₆acyloxy or C₂-C₆acyloxyC₁-C₆alkoxy;
R³ is hydrogen;
R⁴ is methyl;
R⁵ is methyl;
R⁶ is C₁-C₆alkyl, phenyl or heteroaryl, wherein the heteroaryl moiety is a 5- or 6-membered aromatic ring which comprises 1 or 2 heteroatoms individually selected from N, O and S, and wherein the phenyl and heteroaryl moieties are optionally substituted by 1 or 2 substituents, which may be the same or different, selected from Rₐ;
R⁷ is phenyl, phenoxy or heteroaryl, wherein the heteroaryl moiety is a 5- or 6-membered aromatic ring which comprises 1 or 2 nitrogen atoms (e.g. pyridyl), and wherein the phenyl and heteroaryl moieties are optionally substituted by 1 or 2 substituents, which may be the same or different, selected from Rₐ; and
R⁸ is halogen.

Even more preferably, R¹ is hydrogen or formyl;
R² is hydroxyl or C₂-C₆acyloxy;
R³ is hydrogen;
R⁴ is methyl;
R⁵ is methyl;
R⁶ is C₁-C₆alkyl, phenyl or heteroaryl, wherein the heteroaryl moiety is a 5- or 6-membered aromatic ring which comprises 1 or 2 heteroatoms individually selected from N, O and S, and wherein the phenyl and heteroaryl moieties are optionally substituted by 1 or 2 substituents, which may be the same or different, selected from R⁸;
R⁷ is phenyl, phenoxy or heteroaryl, wherein the heteroaryl moiety is a 5- or 6-membered aromatic ring which comprises 1 or 2 nitrogen atoms (e.g. pyridyl), and wherein the phenyl and heteroaryl moieties are optionally substituted by 1 or 2 substituents, which may be the same or different, selected from R⁸; and
R⁸ is halogen.

Still more preferably, R¹ is hydrogen or formyl;
R² is hydroxyl or C₂-C₆acyloxy;
R³ is hydrogen;
R⁴ is methyl;
R⁵ is methyl;
R⁶ is isopropyl, phenyl or 4-fluorophenyl; and
R⁷ is phenyl, phenoxy or 4-fluorophenyl.

In a further set of preferred embodiments, R¹ is hydrogen or formyl;
R² is hydroxyl or isobutyryloxymethoxy;
R³ is hydrogen;
R⁴ is methyl;
R⁵ is methyl;
R⁶ is phenyl or 4-fluorophenyl; and
R⁷ is phenyl or 4-fluorophenyl.
   Preferably, the compound according to formula (I) is selected from:

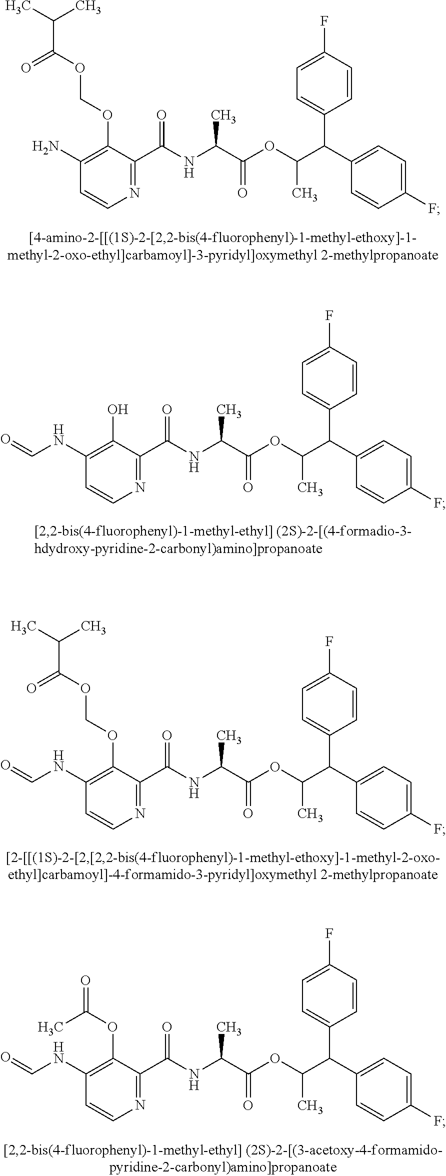 [SMILES: CC(C)C(=O)OCOc1c(N)ccnc1C(=O)N[C@@H](C)C(=O)OC(C)C(c1ccc(F)cc1)c1ccc(F)cc1 ; CC(OC(=O)[C@H](C)NC(=O)c1nccc(NC=O)c1O)C(c1ccc(F)cc1)c1ccc(F)cc1 ; CC(C)C(=O)OCOc1c(NC=O)ccnc1C(=O)N[C@@H](C)C(=O)OC(C)C(c1ccc(F)cc1)c1ccc(F)cc1 ; CC(=O)Oc1c(NC=O)ccnc1C(=O)N[C@@H](C)C(=O)OC(C)C(c1ccc(F)cc1)c1ccc(F)cc1]

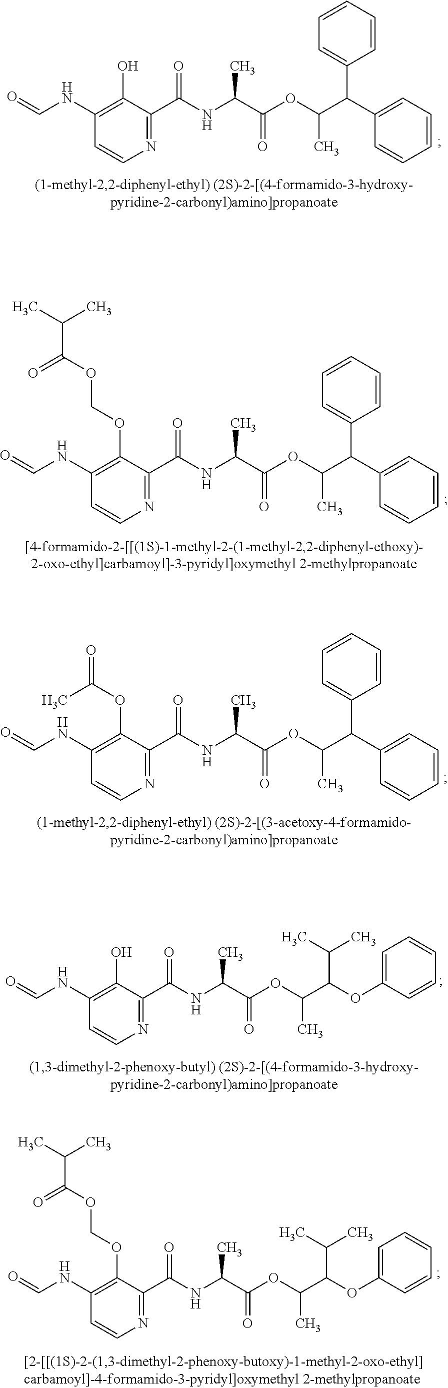 [SMILES: CC(OC(=O)[C@H](C)NC(=O)c1nccc(NC=O)c1O)C(c1ccccc1)c1ccccc1 ; CC(C)C(=O)OCOc1c(NC=O)ccnc1C(=O)N[C@@H](C)C(=O)OC(C)C(c1ccccc1)c1ccccc1 ; CC(=O)Oc1c(NC=O)ccnc1C(=O)N[C@@H](C)C(=O)OC(C)C(c1ccccc1)c1ccccc1 ; CC(C)C(Oc1ccccc1)C(C)OC(=O)[C@H](C)NC(=O)c1nccc(NC=O)c1O ; CC(C)C(=O)OCOc1c(NC=O)ccnc1C(=O)N[C@@H](C)C(=O)OC(C)C(Oc1ccccc1)C(C)C]

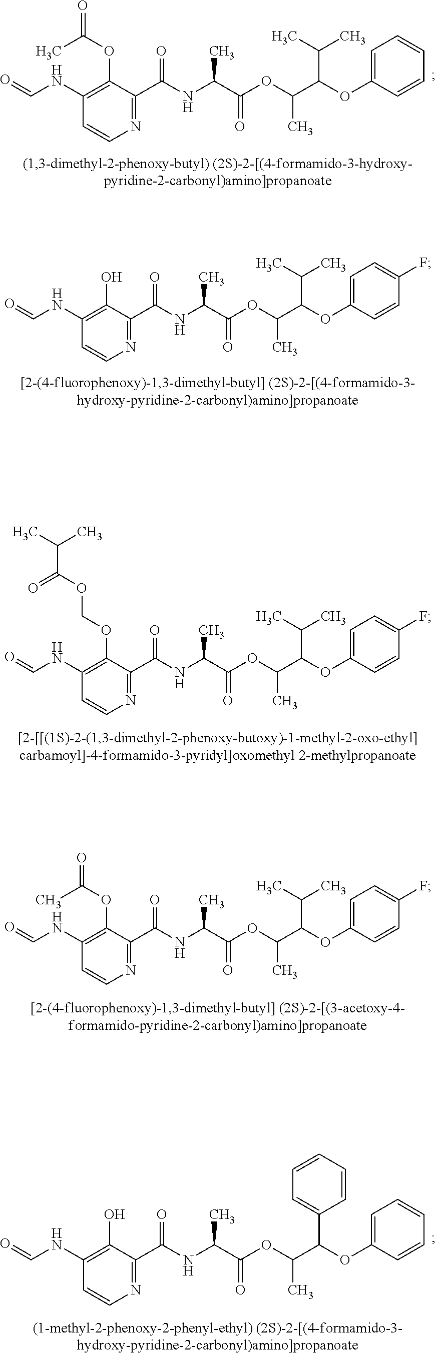 [SMILES: CC(=O)Oc1c(NC=O)ccnc1C(=O)N[C@@H](C)C(=O)OC(C)C(Oc1ccccc1)C(C)C ; CC(C)C(Oc1ccc(F)cc1)C(C)OC(=O)[C@H](C)NC(=O)c1nccc(NC=O)c1O ; CC(C)C(=O)OCOc1c(NC=O)ccnc1C(=O)N[C@@H](C)C(=O)OC(C)C(Oc1ccc(F)cc1)C(C)C ; CC(=O)Oc1c(NC=O)ccnc1C(=O)N[C@@H](C)C(=O)OC(C)C(Oc1ccc(F)cc1)C(C)C ; CC(OC(=O)[C@H](C)NC(=O)c1nccc(NC=O)c1O)C(Oc1ccccc1)c1ccccc1]

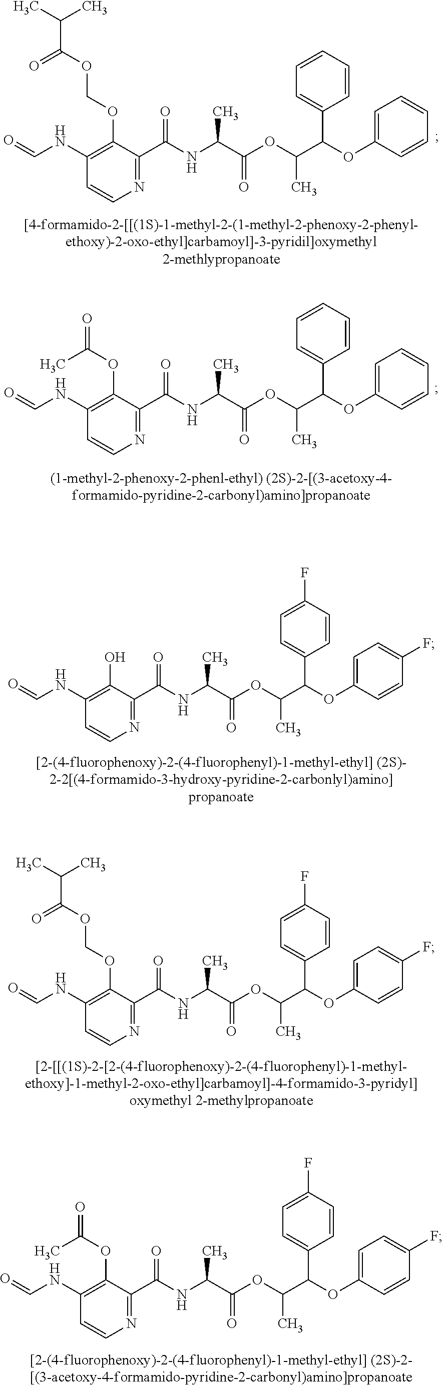 [SMILES: CC(C)C(=O)OCOc1c(NC=O)ccnc1C(=O)N[C@@H](C)C(=O)OC(C)C(Oc1ccccc1)c1ccccc1 ; CC(=O)Oc1c(NC=O)ccnc1C(=O)N[C@@H](C)C(=O)OC(C)C(Oc1ccccc1)c1ccccc1 ; CC(OC(=O)[C@H](C)NC(=O)c1nccc(NC=O)c1O)C(Oc1ccc(F)cc1)c1ccc(F)cc1 ; CC(C)C(=O)OCOc1c(NC=O)ccnc1C(=O)N[C@@H](C)C(=O)OC(C)C(Oc1ccc(F)cc1)c1ccc(F)cc1 ; CC(=O)Oc1c(NC=O)ccnc1C(=O)N[C@@H](C)C(=O)OC(C)C(Oc1ccc(F)cc1)c1ccc(F)cc1]

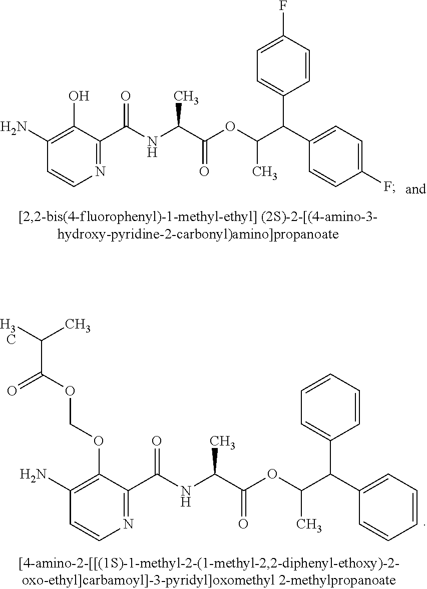 [SMILES: CC(OC(=O)[C@H](C)NC(=O)c1nccc(N)c1O)C(c1ccc(F)cc1)c1ccc(F)cc1 ; CC(C)C(=O)OCOc1c(N)ccnc1C(=O)N[C@@H](C)C(=O)OC(C)C(c1ccccc1)c1ccccc1]

More preferably, the compound according to formula (I) is selected from:
[2,2-bis(4-fluorophenyl)-1-methyl-ethyl] (2S)-2-[(4-formamido-3-hydroxy-pyridine-2-carbonyl)amino]propanoate;
[2-[[(1S)-2-[2,2-bis(4-fluorophenyl)-1-methyl-ethoxy]-1-methyl-2-oxo-ethyl]carbamoyl]-4-formamido-3-pyridyl]oxymethyl 2-methylpropanoate;
[4-amino-2-[[(1 S)-2-[2,2-bis(4-fluorophenyl)-1-methyl-ethoxy]-1-methyl-2-oxo-ethyl]carbamoyl]-3-pyridyl]oxymethyl 2-methylpropanoate;
[4-formamido-2-[[(1S)-1-methyl-2-(1-methyl-2,2-diphenyl-ethoxyl)-2-oxo-ethyl]carbamoyl]-3-20 pyridyl]oxymethyl 2-methylpropanoate;
[2,2-bis(4-fluorophenyl)-1-methyl-ethyl] (2S)-2-[(4-amino-3-hydroxy-pyridine-2-carbonyl) amino]propanoate; and
[4-amino-2-[[(1S)-1-methyl-2-(1-methyl-2,2-diphenyl-ethoxyl)-2-oxo-ethyl]carbamoyl]-3-pyridyl]oxymethyl 2-methylpropanoate.

Compounds of the present invention can be made as shown in the following schemes, in which, unless otherwise stated, the definition of each variable is as defined above for a compound of formula (I).

The compounds of formula (I) according to the invention, wherein R¹, R², R³, R⁴, R⁵, R⁶ and R⁷ are as defined for formula (I), can be obtained by transformation of a compound of formula (II), wherein R¹ and R² are as defined for formula (I) and R⁹ is hydroxyl, halogen or C₁-C₆alkoxy, with a compound of formula (III), wherein R³, R⁴, R⁵, R⁶ and R⁷ are as defined for formula (I), and a base or a peptide coupling reagent. This is shown in Scheme 1 below.

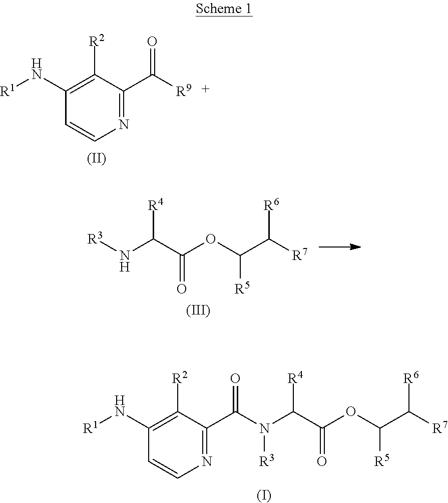

Alternatively, the compounds of formula (I), wherein R¹, R², R³, R⁴, R⁵, R⁶ and R⁷ are as defined for formula (I), can be obtained by transformation of a compound of formula (IV), wherein R¹, R², R³, and R⁴ are as defined for formula (I) and R⁹ is hydroxyl, halogen or C₁-C₆alkoxy, with a compound of formula (V), wherein R⁵, R⁶ and R⁷ are as defined for formula (I), and an acid or a base. This is shown in Scheme 2 below.

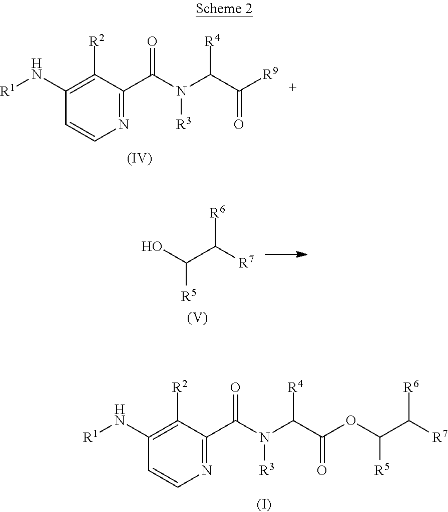

The compounds of formula (II), wherein R¹ and R² are as defined for formula (I) and R⁹ is hydroxyl, halogen or C₁-C₆alkoxy, can be obtained by transformation of a compound of formula (VI), wherein R² is as defined for formula (I) and R⁹ is hydroxyl, halogen or C₁-C₆alkoxy, with a carboxylic acid and an acid anhydride or a carboxylic acid chloride. This is shown in Scheme 3 below.

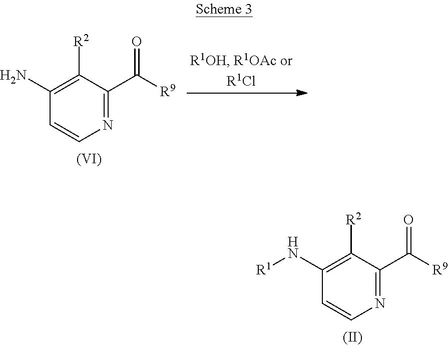

The compounds of formula (III), wherein R³, R⁴, R⁵, R⁶ and R⁷ are as defined for formula (I), can be obtained by transformation of a compound of formula (VII), wherein R³ and R⁴ are as defined for formula (I), R⁹ is hydroxyl, halogen or C₁-C₆alkoxy and R¹⁰ is C₁-C₆alkylcarbonyl or C₁-C₆alkoxycarbonyl, with a compound of formula (V), wherein R⁵, R⁶ and R⁷ are as defined for formula (I), and an acid or a base. This is shown in Scheme 4 below.

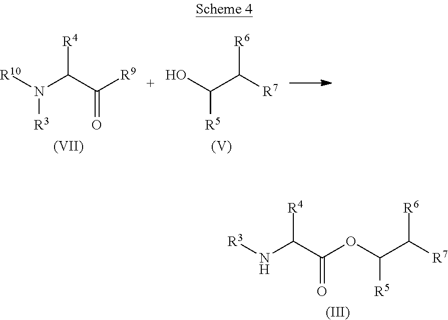

The compounds of formula (IV), wherein R¹, R², R³, and R⁴ are as defined for formula (I) and R⁹ is hydroxyl, halogen or C₁-C₆alkoxy, can be obtained by transformation of a compound of formula (VIII), wherein R², R³, and R⁴ are as defined for formula (I) and R⁹ is hydroxyl, halogen or C₁-C₆alkoxy, with a carboxylic acid and an acid anhydride or a carboxylic acid chloride. This is shown in Scheme 5 below.

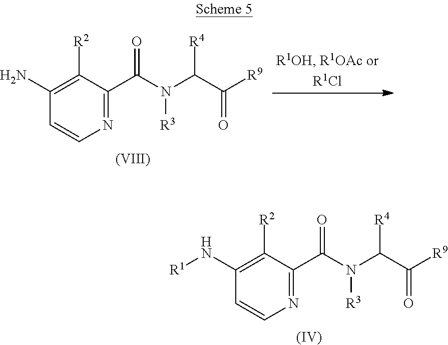

The compounds of formula (VI), wherein R² is as defined for formula (I) and R⁹ is hydroxyl, halogen or C₁-C₆alkoxy can be obtained by transformation of a compound of formula (IX), wherein R² is as defined for formula (I) and R⁹ is hydroxyl, halogen or C₁-C₆alkoxy, under reductive conditions, e.g. catalytic hydrogenation. This is shown in Scheme 6 below.

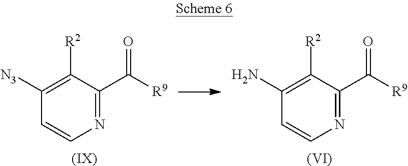

The compounds of formula (VIII), wherein R², R³, and R⁴ are as defined for formula (I) and R⁹ is hydroxyl, halogen or C₁-C₆alkoxy, can be obtained by transformation of a compound of formula (X), wherein R², R³, and R⁴ are as defined for formula (I) and R⁹ is hydroxyl, halogen or C₁-C₆alkoxy, under reductive conditions, e.g. catalytic hydrogenation. This is shown in Scheme 7 below.

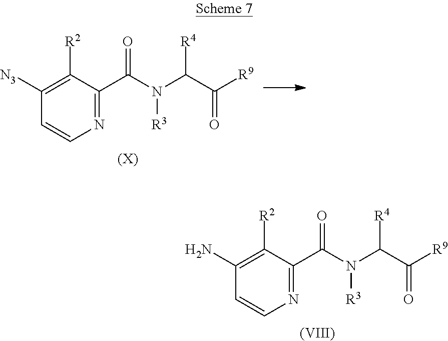

Alternatively, the compounds of formula (I), wherein R¹, R², R³, R⁴, R⁵, R⁶ and R⁷ are as defined for formula (I), can be obtained by transformation of a compound of formula (XI), wherein R², R³, R⁴, R⁵, R⁶ and R⁷ are as defined for formula (I), with a carboxylic acid and an acid anhydride or a carboxylic acid chloride. This is shown in Scheme 8 below.

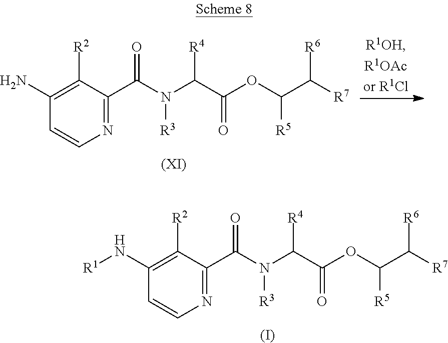

The compounds of formula (XI), wherein R², R³, R⁴, R⁵, R⁶ and R⁷ are as defined for formula (I), can be obtained by transformation of a compound of formula (XII), wherein R², R³, R⁴, R⁵, R⁶ and R⁷ are as defined for formula (I), under reductive conditions, e.g. catalytic hydrogenation. This is shown in Scheme 9 below.

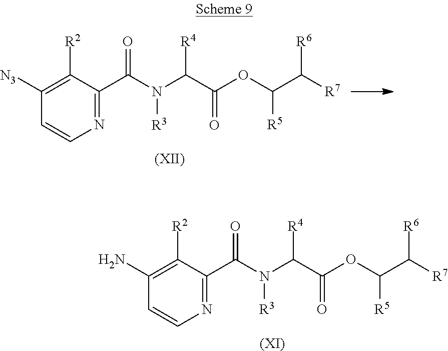

The compounds of formula (XII), wherein R², R³, R⁴, R⁵, R⁶ and R⁷ are as defined for formula (I), can be obtained by transformation of a compound of formula (X), wherein R², R³, and R⁴ are as defined for formula (I) and R⁹ is hydroxyl, halogen or C₁-C₆alkoxy, with a compound of formula (V), wherein R⁵, R⁶ and R⁷ are as defined for formula (I), and with an acid or a base. This is shown in Scheme 10 below.

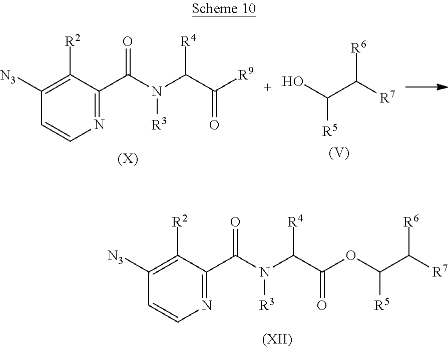

Alternatively, the compounds of formula (XII), wherein R², R³, R⁴, R⁵, R⁶ and R⁷ are as defined for formula (I), can be obtained by transformation of a compound of formula (IX), wherein R² is as defined for formula (I) and R⁹ is hydroxyl, halogen or C₁-C₆alkoxy, with a compound of formula (III), wherein R³, R⁴, R⁵, R⁶ and R⁷ are as defined for formula (I), and with a base or a peptide coupling reagent. This is shown in Scheme 11 below.

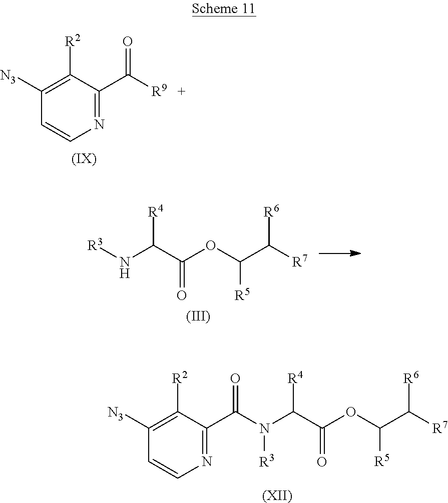

The compounds of formula (I) according to the present invention may possess one, two or three chiral centres at carbon atoms A, B and C as outlined below in formula (A).

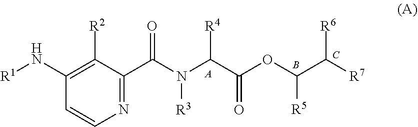

Where there are two chiral centres (A and B), the compounds of formula (I) may exist in various diastereomeric forms, i.e., with (S,S,S)-, (S,S,R)-, (S,R,R)-, (S,R,S)-, (R,R,R)-, (R,R,S)-, (R,S,S)- or (R,S,R)-configurations present at the A, B and C carbons, respectively.

Each of these stereochemical configurations as described for formula (A) may be evident for compounds of formula (I) in relation to the specific combinations of definitions for R¹, R², R³, R⁴, R⁵, R⁶, R⁷ and R⁸for each compound described in Table 1 (a compound of formulae (1.a.1-1.a.128 to (1.y.1-1.y.128)) or a compound of formula (I) described in Table 2 (below).

In some embodiments of the invention, the compound of Formula (I) may be the compound of Formula (IA).

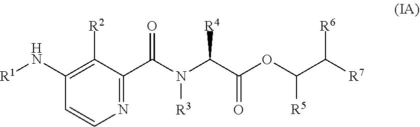

Surprisingly, it has now been found that the novel compounds of formula (I) have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use the compounds of formula (I) as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It is also possible to use compounds of formula (I) as dressing agents for the treatment of plant propagation material, e.g., seeds, such as fruits, tubers or grains, or plant cuttings (e.g., rice), for the protection against fungal infections, as well as against phytopathogenic fungi occurring in the soil. The propagation material can be treated with a composition comprising a compound of formula (I) before planting: seeds, e.g., can be dressed before being sown.

The active ingredients according to the invention can also be applied to grains (coating), either by impregnating the seeds in a liquid formulation or by coating them with a solid formulation. The composition can also be applied to the planting site when the propagation material is being planted, e.g., to the seed furrow during sowing. The invention relates also to such methods of treating plant propagation material and to the plant propagation material so treated.

Furthermore, the compounds according to the present invention can be used for controlling fungi in related areas, for example in the protection of technical materials, including wood and wood related technical products, in food storage, in hygiene management.

In addition, the invention could be used to protect non-living materials from fungal attack, e.g., lumber, wall boards and paint.

The compounds of formula (I) may be, for example, effective against fungi and fungal vectors of disease as well as phytopathogenic bacteria and viruses. These fungi and fungal vectors of disease as well as phytopathogenic bacteria and viruses are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; omamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(bi) toxin); YieldGard Plus® (maize variety that expresses a CryIA(b) and a CryIIIB(bi) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as 8-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-gycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 8-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard 10 (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe SA. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for 30 achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize 35 varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringrensis subsp. kurstaki which brings about tolerance to certain Lepidoptera, include the European corn borer.

The compounds of formula (I) according to the present invention (including any one of compounds described in Table 2 (below)) may be used in controlling or preventing phytopathogenic diseases, especially phytopathogenic fungi (such as Phakopsora pachyrhizi) on soybean plants.

In particular, transgenic soybean plants expressing toxins, for example insecticidal proteins such as delta-endotoxins, e.g. Cry1Ac (Cry1Ac Bt protein). Accordingly, this may include transgenic soybean plants comprising event MON87701 (see U.S. Pat. No. 8,049,071 and related applications and patents, as well as WO 2014/170327 A1 (eg, see paragraph [008] reference to Intacta RR2 PRO™ soybean)), event MON87751 (US. Patent Application Publication No. 2014/0373191) or event DAS-81419 (U.S. Pat. No. 8,632,978 and related applications and patents).

Other transgenic soybean plants may comprise event SYHT0H2—HPPD tolerance (U.S. Patent Application Publication No. 2014/0201860 and related applications and patents), event MON89788—glyphosate tolerance (U.S. Pat. No. 7,632,985 and related applications and patents), event MON87708—dicamba tolerance (U.S. Patent Application Publication No. US 2011/0067134 and related applications and patents), event DP-356043-5—glyphosate and ALS tolerance (U.S. Patent Application Publication No. US 2010/0184079 and related applications and patents), event A2704-12—glufosinate tolerance (U.S. Patent Application Publication No. US 2008/0320616 and related applications and patents), event DP-305423-1—ALS tolerance (U.S. Patent Application Publication No. US 2008/0312082 and related applications and patents), event A5547-127—glufosinate tolerance (U.S. Patent Application Publication No. US 2008/0196127 and related applications and patents), event DAS-40278-9—tolerance to 2,4-dichlorophenoxyacetic acid and aryloxyphenoxypropionate (see WO 2011/022469, WO 2011/022470, WO 2011/022471, and related applications and patents), event 127—ALS tolerance (WO 2010/080829 and related applications and patents), event GTS 40-3-2—glyphosate tolerance, event DAS-88416-4-2,4-dichlorophenoxyacetic acid and glufosinate tolerance, event FG72—glyphosate and isoxaflutole tolerance, event BPS-CV127-9—ALS tolerance and GU262—glufosinate tolerance or event SYHT04R—HPPD tolerance.

Under certain circumstances, compounds of Formula (I) according to the present invention when used in controlling or preventing phytopathogenic diseases, especially phytopathogenic fungi (such as Phakopsora pachyrhizi) on soybean plants (in particular any of the transgenic soybean plants as described above), may display a synergistic interaction between the active ingredients.

Additionally, to date, no cross-resistance has been observed between the compounds of Formula (I) (including any one of compounds described in Table 2 (below)) and the current fungicidal solutions used to control Phakopsora pachyrhizi.

Indeed, fungicidal-resistant strains of Phakopsora pachyrhizi have been reported in the scientific literature, with strains resistant to one or more fungicides from at least each of the following fungicidal mode of action classes being observed: sterol demethylation-inhibitors (DMI), quinone-outside-inhibitors (QoI) and succinate dehydrogenase inhibitors (SDHI). See for example: “of Phakopsora pachyrhizi towards quinone-outside-inhibitors and demethylation-inhibitors, and corresponding resistance mechanisms.” Schmitz H K at al, Pest Manag Sci (2014) 70: 378-388; “First detection of a SDH variant with reduced SDHI sensitivity in Phakopsora pachyrhizi” Simões K et al, J Plant Dis Prot (2018) 125: 21-2; “Competitive fitness of Phakopsora pachyrhizi isolates with mutations in the CYP51 and CYTB genes.” Klosowski A C et al, Phytopathology (2016) 106: 1278-1284; “Detection of the F129L mutation in the cytochrome b gene in Phakopsora pachyrhizi.” Klosowski A C et al, Pest Manag Sci (2016) 72:1211-1215.

Thus, in a preferred embodiment, the compounds of Formula (I) (including any one of compounds described in Table 2 (below)), or fungicidal compositions according to the present invention comprising a compound of Formula (I), are used to control Phakopsora pachyrhizi which are resistant to one or more fungicides from any of the following fungicidal MoA classes: sterol demethylation-inhibitors (DMI), quinone-outside-inhibitors (QoI) and succinate dehydrogenase inhibitors (SDHI).

The compounds of Formula (I) (including any one of described in Table 2 (below)) or fungicidal compositions according to the present invention comprising a compound of Formula (I) may be used in controlling or preventing phytopathogenic diseases, especially phytopathogenic fungi (such as Phakopsora pachyrhizi) on soy bean plants. In particular, there are known in the scientific literature certain Elite soybean plant varieties where R-gene stacks, conferring a degree of immunity or resistance to specific Phakopsora pachyrhizi, have been introgressed in the plant genome, see for example: “Fighting Asian Soybean Rust”, Langenbach C, et al, Front Plant Science 7(797) 2016).

An elite plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minn., USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R⁶; S73-S8; and S78-G6 (Syngenta Seeds, Henderson, Ky., USA); Richer (Northstar Seed Ltd. Alberta, Calif.); 14RD62 (Stine Seed Co. Ia., USA); or Armor 4744 (Armor Seed, LLC, Ar., USA).

Thus, in a further preferred embodiment, the compounds of Formula (I) (including any one of compounds described in Table 2 (below)), or fungicidal compositions according to the present invention comprising a compound of Formula (I), are used to control Phakopsora pachyrhizi, (including fungicidally-resistant strains thereof, as outlined above) on Elite soybean plant varieties where R-gene stacks, conferring a degree of immunity or resistance to specific Phakopsora pachyrhizi, have been been introgressed in the plant genome. Numerous benefits may be expected to ensue from said use, e.g. improved biological activity, an advantageous or broader spectrum of activity (inc. sensitive and resistant strains of Phakopsora pachyrhizi), an increased safety profile, improved crop tolerance, synergistic interactions or potentiating properties, improved onset of action or a longer lasting residual activity, a reduction in the number of applications and/or a reduction in the application rate of the compounds and compositions required for effective control of the phytopathogen (Phakopsora pachyrhizi), thereby enabling beneficial resistance-management practices, reduced environmental impact and reduced operator exposure.

Additionally, to date, no cross-resistance has been observed between the compounds of Formula (I) (including any one of compounds described in Table 2 (below)) and any fungicidal solutions used to control phytopathogenic fungi such as Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocysis spp, Cerospora spp. including C. arachidicola, Cercospordium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichracearum, Eutypa lata, Fusarium spp. including F. culmorum, F. graminearum, F. langsethiae, F. moniliforme, F. oxysporum, F. proliferatum, F. subgfudinans, F. solani, Gaeumannomyces graminis, Gibberella fujikuroi, Gloeodes pomigena, Gloeosponum musarum, Glomerella cingulate, Guignardia bidwellii, Gymnosporangium juniperi-virgnianae, Helminhosponum spp, Hemileia spp, Histoplasma spp. including H. capsulatum, Laetisana fuciformis, Leptogaphium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phelinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudoceroosporella herpoftihoides, Pseudomonas spp, Pseudoperonospora spp. including P. cubensis, P. humuli, Pseudopeziza kracheiphila, Puccinia Spp. including P. hordei, P. recondita, P. striiformis, P. triticina, Pyrenopeziza spp, Pyrenophora spp, Pyriculaia spp. including P. oryzae, Pythium spp. including P. ultimum, Ramulana spp, Rhizoctonia spp, Rhizomucor pusillus, Rhizopus arrhizus, Rhynchosponum spp, Scedosponum spp. including S. apiospermum and S. proliflcans, Schizothyrum pomi, Sclerohnia spp, Scleroum spp, Septoria spp, including S. nodorum, S. tritici, Sphaerotheca maculars, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Ventuia spp. including V. inaequalis, Verticillium spp, and Xanthomonas spp., in particular, Zymoseptoia tritici, Puccinia recondita, Puccinia sfriiformis, Erysiphe graminis, Uncinula necator, Sphaerotheca fuliginea, Leveillula taurica, Phakopsora pachyrhizi, Pyriculaia oryzae, Altemana solani, Altemana altemata, Mycosphaerella fijiensis, Colletotichum lagenarium, Didymella bryoniae, Ascochyta pisii, Vericillium dahliae, Pyrenophora teres, Cercospora beticola, Ramularia collo-cygni, Botrytis cinerea, Sclerohnia sclerotiorum, Monilinia laxa, Monographaella nivalis and Ventuia inaequalis.

Indeed, fungicidal-resistant strains in any of the species as outlined above have been reported in the scientific literature, with strains resistant to one or more fungicides from at least one of the following fungicidal mode of action classes: quinone-outside-inhibitors (QoI), quinone-inside-inhibitors (QiI), succinate dehydrogenase inhibitors (SDHI) and sterol demethylation-inhibitors (DMI). Such fungicidal-resistant strains may contain:
A mutation in the mitochondrial cytochrome b gene conferring resistance to Q₀ inhibitors, wherein the mutation is G143A, F129L or G137R. See for example: Gisi et al., Pest Manag Sci 56, 833-841, (2000), Lucas, Pestic Outlook 14(6), 268-70 (2003), Fraaije et al., Phytopathol 95(8), 933-41 (2005), Sierotzki et al., Pest Manag Sci 63(3), 225-233 (2007), Semar et al., Journal of Plant Diseases and Protection (3), 117-119 (2007); and Pasche et al., Crop Protection 27(3-5), 427-435 (2008).
A mutation in the mitochondrial cytochrome b gene conferring resistance to Qi inhibitors, wherein the mutation is G37A/C/D/SN. See for example: Meunier et al., Pest Manag Sci 2019; 75: 2107-2114.
A mutation in the genes encoding the SdhB,C,D subunits conferring resistance to SDHI inhibitors wherein the mutation is in the following major pathogens:
   Botrytis cinerea: B-P225H/L/T/Y/F, B-N230I, B-H272L/Y/R, C-P80H/L, C-N87S;
   Alternaria solani: B-H278R/Y, C-H134R/Q, D-D123E, D-H133R and C-H134R;
   Zymoseptoria tritici: sdhB: N225T, N225I, R265P, T2681, T268A. In sdhC: T79N, T791, W80S, W80A, A84F, N86S, N86A, P127A, R151MS/IT/G, R151S, R151T, H152R/Y, V166M, T168R. In sdhD: 150F, M114V, D129G, T20P+K186R;
   Pyrenophora teres: In sdhB: S66P, N2351, H277Y. In sdhC: K49E, R64K, N75S, G79R, H134R, S135R. In sdhD: D124E, H134R, G138V, D145G;
   Ramularia collo-cygni: In sdhB: N224T, T2671. In sdhC: N87S, G91R, H146R/L, G171D, H153R;
   Phakospora pachyrhizi: C-186F;
   Sclerotinia sclerotiorum: In sdhB: H273Y. In sdhC: G91R, H146R. In sdhD: T108K, H132R, G150R.
      Major source of information is www.frac.info, Sierotzki and Scalliet Phytopathology (2013) 103(9): 880-887 and Simões et al., J Plant Dis Prot (2018) 125: 21-2.
A mutation or combination of mutations in the CYP51 gene conferring resistance to DMI inhibitors wherein the mutations are: L50S, D134G, V136A/C, Y137F, S188N, A379G, I381V, deletion 459-460, Y461H/S, N513K, S524T. Major source of information is www.frac.info, Cools et al., Plant Pathol (2013) 62: 36-42 and Schmitz H K et al., Pest Manag Sci (2014) 70: 378-388.

Thus, in a preferred embodiment, the compounds of Formula (I) (Including any one of compounds described in Table 2 (below)), or fungicidal compositions according to the present invention comprising a compound of Formula (I), are used to control fungal strains which are resistant to one or more fungicides from any of the following fungicidal MoA classes: quinone-outside-inhibitors (QoI), quinone-inside-inhibitors (QiI), succinate dehydrogenase inhibitors (SDHI) and sterol demethylation-inhibitors (DMI).

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 15th Ed., British Crop Protection Council 2009.

The compounds of formula (I) may be used in unmodified form or, preferably, together with the adjuvants conventionally employed in the art of formulation. To this end, they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula (I) are normally used in the form of compositions and can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of (fungicidal) compositions for controlling or protecting against phytopathogenic microorganisms, comprising as active ingredient at least one compound of formula (I) or of at least one preferred individual compound as above-defined, in free form or in agrochemically usable salt form, and at least one of the above-mentioned adjuvants.

The invention provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably, said composition may comprise at least one or more pesticidally active compounds, for example an additional fungicidal active ingredient in addition to the compound of formula (I).

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients also include the following: petroleum oils, 1,1-bis(4-chloro-phenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromo-cyclen, bromophos, bromopropylate, buprofezin, butocarboxim, butoxycarboxim, butylpyridaben, calcium polysulfide, camphechlor, carbanolate, carbophenothion, cymiazole, chino-methionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dino-penton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fen-pyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, procionol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperia camea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinemema bibionis, Steinemema carpocapsae, Steinemema feltiae, Steinemema glaseri, Steinemema riobrave, Steinemema riobravis, Steinemema scapterisci, Steinemema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-8-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, dispariure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure Ill, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure Bi, trimedlure B2, trimedlure C, trunc-call, 2-(octylthio)-ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)-ethane, 1,2-dichloropropane with 1,3-dichoropropene, 1-bromo-2-chloroethane, 2,2,2-trichloro-1-(3,4-dichloro-phenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethyl-carbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone Ill, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nomicotine, O-5-dichloro-4-iodophenyl O-ethyl ethylphosphonothioate, O,O-diethyl O-4-methyl-2-oxo-2H-chromen-7-yl phosphorothioate, O,O-diethyl O-8-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene Ill, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla), schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, and meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothio-phene 1,1-dioxide, 3-(4-chlorophenyl)-5-methylrhodanine, 5-methyl-8-thioxo-1,3,5-thiadiazinan-3-ylacetic acid, 6-isopentenylaminopurine, 2-fluoro-N-(3-methoxyphenyl)-9H-purin-6-amine, benclothiaz, cytokinins, DCIP, furfural, isamidofos, kinetin, Myrothecium verrucaria composition, tetrachlorothiophene, xylenols, zeatin, potassium ethytxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoro-acetate, thallium sulfate, warfarin, 2-(2-butoxyethoxy)-ethyl piperonylate, 5-(1,3-benzodioxol-5-yl)-3-hexylcyclohex-2-enone, famesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, mercuric oxide, thiophanate-methyl, azaconazole, bitertanol, bromuconazole, cyproconazole, difenoconazole, diniconazole, epoxicon-azole, fenbuconazole, fluquinconazole, flusilazole, flutriafol, furametpyr, hexaconazole, imazalil, imiben-con-azole, ipconazole, metconazole, myclobutanil, paclobutriazole, pefurazoate, penconazole, prothioconazole, pyrifenox, prochloraz, propiconazole, pyrisoxazole, simeconazole, tebucon-azole, tetraconazole, triadimefon, triadime-nol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, meta-laxyl, R-metalaxyl, ofurace, oxadixyl, carbendazim, debacarb, fuberidazole, thiaben-dazole, chlozolinate, dichlozoline, myclozoline, procymi-done, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metomi-nostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolciofos-methyl, anilazine, benthiavalicarb, blasticidin-S, chloroneb, chloro-tha-io-nil, cyflufenamid, cymoxanil, cyclobutrifluram, diclocymet, diclomezine, dicloran, diethofencarb, dimetho-morph, flumorph, dithianon, ethaboxam, etridiazole, famoxa-done, fenamidone, fenoxanil, ferimzone, fluazinam, fluopicolide, flusulfamide, fluxapyroxad, fenhexamid, fos-etyl-aluminium, hymexazol, iprovalicarb, cyazofamid, methasulfo-carb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trfluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-8-methyl-5-phenyl-pyridazine-3-carbonitrile, (R)-3-(difluoromethyl)-1-methyl-N-[1,1,3-timethylindan-4-yl]pyrazole-4-carboxamide, 4-(2-bromo-4-fluoro-phenyl)-N-(2-chloro-8-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), lvbenmixianan, dichlobentiazox, mandestrobin, 3-(4,4-difluoro-3,4-dihydro-3,3-dimethylisoquinolin-1-yl)quinolone, 2-[2-fluoro-8-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl-4-piperidyl]thiazol-4-yl]-4,5-dihydroisoxazol-5-yl]-3-chloro-phenyl] methanesulfonate, but-3-ynyl N-[6-[[(Z)-[(1-methyltetriazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-8-methyl-5-phenyl-4-(2,4,6-trifluorophenyl)pyridazine, pyridachlometyl, 3-(difluoromethyl)-1-methyl-N-[1,1,3-timethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chloropheny)pyrazol-3-yl]oxy-2-methoxyimino-N,3-dimethyl-pent-3-enamide, florylpicoxamid, fenpicoxamid, tebufloquin, ipflufenoquin, quinofumelin, isofetamid, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flutianil, fluopimomide, pyrapropoyne, picarbutrazox, 2-(difluoromethyl)-N-(3-ethyl-.11-dimethyl-indan-4-y)pyridine-3-carboxamide, 2-(difluoromethyl)-N-((3R)-1,1,3-trimethylindan-4-yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1,1,3-trimethylindan-4-y) pyridine-3-carboxamide, α-(1,1-dimethylethyl)-α-[4′-(trifluoromethoxy)[1,1′-biphenyl]-4-yl]-5-pyrimidinemethanol, fluoxapiprolin, enoxastrobin, 4-[[6-[2-(2,4-difluorophenyl)-.11-difluoro-2-hydroxy-3-(1,2,4-triazol-1-yl)propyl]-3-pyridyl]oxy] benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, N-octyl-N′-[2-(octylamino)ethyl]ethane-1,2-diamine, N′-[5-bromo-2-methyl-8-[(1S)-1-methyl-2-propoxy-ethoxy]-3-pyridyl]-N-ethyl-N-methyl-formamidine, N′-[5-bromo-2-methyl-8-[(1R)-1-methyl-2-propoxy-ethoxy]-3-pyridyl]-N-ethyl-N-methyl-formamidine, N′-[5-bromo-2-methyl-8-(1-methyl-2-propoxy-ethoxyl)-3-pyridyl]-N-ethyl-N-methyl-formamidine, N′-[5-chloro-2-methyl-8-(1-methyl-2-propoxy-ethoxyl)-3-pyridyl]-N-ethyl-N-methyl-formamidine, N′-[5-bromo-2-methyl-8-(1-methyl-2-propoxy-ethoxyl)-3-pyridyl]-N-isopropyl-N-methyl-formamidine (these compounds may be prepared from the methods described in WO2015/155075); N′-[5-bromo-2-methyl-8-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[2-trifluoromethyl)oxetan-2-yl]phenyl]-N-methyl-formamidine, N-ethyl-N′-[5-methoxy-2-methyl-4-[2-trifluoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethylbutyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trfluoro-3,3-dimethyl-isoquinoline, 4,4-difluoro-3,3-dimethyl-1-(6-methylpyrazolo[1,5-a]pyridin-3-y)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxyl)-2-(trifluoromethyl)-3-pyridyl]-1-(1,2,4-triazol-1-y)propan-2-ol (this compound may be prepared from the methods described in WO 2017/029179); 2-[6-(4-bromophenoxyl)-2-(trifluoromethyl)-3-pyridyl]-1-(1,2,4-triazol-1-y)propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-8-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281)N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxyl)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazoyl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-y]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N—[(Z)-methoxyiminomethyl]-4-[5-(trifluoromethyl)-1,2,4-oxadiazol-3-yl]benzamide, N—[N-methoxy-C-methyl-carbonimidoyl]-4-[5-(trifluoromethyl)-1,2,4-oxadiazol-3-yl]benzamide (these compounds may be prepared from the methods described in WO 2018/202428).

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO-9615121 and also with anthelmintic active cyclic depsipeptides such as those described in WO-9611945, WO-9319053, WO-9325543, EP-626375, EP-382173, WO-9419334, EP-382173, and EP-503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacioprid and the like.

The compounds of the invention may be used in combination with terpene alkaloids, for example those described in WO 95/19363 or WO 04/72086, particularly the compounds disclosed therein.

Other examples of such biologically active compounds that the compounds of the invention may be used in combination with include but are not restricted to the following: Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraciofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, coethocarb, ethiofencarb, fenoxycarb, fenthiocarb, furathiocarb, HCN-801, isoprocarb, indoxacarb, methiocarb, methomyl, 5-methyl-m-cumenylbutyryl(methy)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethy)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycoprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacioden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195,RH-0345, RH-2485, RYI-210, S-1283, S-1833, SI-8601, silafluofen, silomadine, spinosad, tebufenpyrad, tetradifon, tetranactin, thiacioprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, cenbuterol, omeprazole, tiamulin, benazepril, pyriprole, cefquinome, florfenicol, buserelin, cefovecin, tulathromycin, ceftiour, carprofen, metaflumizone, praziquarantel, tricabendazole.

Another aspect of invention is related to the use of a compound of formula (I) or of a preferred individual compound as above-defined, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g., harvested food crops, or non-living materials by insects or by phytopathogenic microorganisms, preferably fungal organisms.

A further aspect of invention is related to a method of controlling or preventing an infestation of plants, e.g., useful plants such as crop plants, propagation material thereof, e.g. seeds, harvested crops, e.g. harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g., in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula (I) per kg of seed, preferably from 0.01 to 10 g per kg of seed are generally sufficient.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (VS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WVP) or any technically feasible formulation in combination with agriculturally acceptable adjuvants.

Such compositions may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g., as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) together with component (B) and (C), and optionally other active agents, particularly microbicides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Table 1 below illustrates examples of individual compounds of formula (I) according to the invention.

TABLE 1
Individual compounds of formula (I)
according to the invention

| Cpd No. | R⁵ | R⁶ | R⁷ |
|---|---|---|---|
| 1 | CH₃ | CH₃ | Ph |
| 2 | CH₃ | CH₃ | 4-F-Ph |
| 3 | CH₃ | CH₃ | F-Cl-Ph |
| 4 | CH₃ | CH₃ | pyrid-3-yl |
| 5 | CH₃ | CH₃ | Ph-O |
| 6 | CH₃ | CH₃ | 4-F-Ph-O |
| 7 | CH₃ | CH₃ | 4-Cl-Ph-O |
| 8 | CH₃ | CH₃ | pyrid-2-yl-O |
| 9 | CH₃ | CH₃CH₂ | Ph |
| 10 | CH₃ | CH₃CH₂ | 4-F-Ph |
| 11 | CH₃ | CH₃CH₂ | 4-Cl-Ph |
| 12 | CH₃ | CH₃CH₂ | pyrid-3-yl |
| 13 | CH₃ | CH₃CH₂ | Ph-O |
| 14 | CH₃ | CH₃CH₂ | 4-F-Ph-O |
| 15 | CH₃ | CH₃CH₂ | 4-Cl-Ph-O |
| 16 | CH₃ | CH₃CH₂ | pyrid-2-yl-O |
| 17 | CH₃ | (CH₃)₂CH | Ph |
| 18 | CH₃ | (CH₃)₂CH | 4-F-Ph |
| 19 | CH₃ | (CH₃)₂CH | 4-Cl-Ph |
| 20 | CH₃ | (CH₃)₂CH | pyrid-3-yl |
| 21 | CH₃ | (CH₃)₂CH | Ph-O |
| 22 | CH₃ | (CH₃)₂CH | 4-F-Ph-O |
| 23 | CH₃ | (CH₃)₂CH | 4-Cl-Ph-O |
| 24 | CH₃ | (CH₃)₂CH | pyrid-2-yl-O |
| 25 | CH₃ | cyclopropyl | Ph |
| 26 | CH₃ | cyclopropyl | 4-F-Ph |
| 27 | CH₃ | cyclopropyl | 4-Cl-Ph |
| 28 | CH₃ | cyclopropyl | pyrid-3-yl |
| 29 | CH₃ | cyclopropyl | Ph-O |
| 30 | CH₃ | cyclopropyl | 4-F-Ph-O |
| 31 | CH₃ | cyclopropyl | 4-Cl-Ph-O |
| 32 | CH₃ | cyclopropyl | pyrid-2-yl-O |
| 33 | CH₃ | Ph | Ph |
| 34 | CH₃ | Ph | 4-F-Ph |
| 35 | CH₃ | Ph | 4-Cl-Ph |
| 36 | CH₃ | Ph | pyrid-3-yl |
| 37 | CH₃ | Ph | Ph-O |
| 38 | CH₃ | Ph | 4-F-Ph-O |
| 39 | CH₃ | Ph | 4-Cl-Ph-O |
| 40 | CH₃ | Ph | pyrid-2-yl-O |
| 41 | CH₃ | 4-F-Ph | Ph |
| 42 | CH₃ | 4-F-Ph | 4-F-Ph |
| 43 | CH₃ | 4-F-Ph | 4-Cl-Ph |
| 44 | CH₃ | 4-F-Ph | pyrid-3-yl |
| 45 | CH₃ | 4-F-Ph | Ph-O |
| 46 | CH₃ | 4-F-Ph | 4-F-Ph-O |
| 47 | CH₃ | 4-F-Ph | 4-Cl-Ph-O |
| 48 | CH₃ | 4-F-Ph | pyrid-2-yl-O |
| 49 | CH₃ | 4-Cl-Ph | Ph |
| 50 | CH₃ | 4-Cl-Ph | 4-F-Ph |
| 51 | CH₃ | 4-Cl-Ph | 4-Cl-Ph |
| 52 | CH₃ | 4-Cl-Ph | pyrid-3-yl |
| 53 | CH₃ | 4-Cl-Ph | Ph-O |
| 54 | CH₃ | 4-Cl-Ph | 4-F-Ph-O |
| 55 | CH₃ | 4-Cl-Ph | 4-Cl-Ph-O |
| 56 | CH₃ | 4-Cl-Ph | pyrid-2-yl-O |
| 57 | CH₃ | pyrid-3-yl | Ph |
| 58 | CH₃ | pyrid-3-yl | 4-F-Ph |
| 59 | CH₃ | pyrid-3-yl | 4-Cl-Ph |
| 60 | CH₃ | pyrid-3-yl | pyrid-3-yl |
| 61 | CH₃ | pyrid-3-yl | Ph-O |
| 62 | CH₃ | pyrid-3-yl | 4-F-Ph-O |
| 63 | CH₃ | pyrid-3-yl | 4-Cl-Ph-O |
| 64 | CH₃ | pyrid-3-yl | pyrid-2-yl-O |
| 65 | CH₃CH₂ | CH₃ | Ph |
| 66 | CH₃CH₂ | CH₃ | 4-F-Ph |
| 67 | CH₃CH₂ | CH₃ | 4-Cl-Ph |
| 68 | CH₃CH₂ | CH₃ | pyrid-3-yl |
| 69 | CH₃CH₂ | CH₃ | Ph-O |
| 70 | CH₃CH₂ | CH₃ | 4-F-Ph-O |
| 71 | CH₃CH₂ | CH₃ | 4-Cl-Ph-O |
| 72 | CH₃CH₂ | CH₃ | pyrid-2-yl-O |
| 73 | CH₃CH₂ | CH₃CH₂ | Ph |
| 74 | CH₃CH₂ | CH₃CH₂ | 4-F-Ph |
| 75 | CH₃CH₂ | CH₃CH₂ | 4-Cl-Ph |
| 76 | CH₃CH₂ | CH₃CH₂ | pyrid-3-yl |
| 77 | CH₃CH₂ | CH₃CH₂ | Ph-O |
| 78 | CH₃CH₂ | CH₃CH₂ | 4-F-Ph-O |
| 79 | CH₃CH₂ | CH₃CH₂ | 4-Cl-Ph-O |
| 80 | CH₃CH₂ | CH₃CH₂ | pyrid-2-yl-O |
| 81 | CH₃CH₂ | (CH₃)₂CH | Ph |
| 82 | CH₃CH₂ | (CH₃)₂CH | 4-F-Ph |
| 83 | CH₃CH₂ | (CH₃)₂CH | 4-Cl-Ph |
| 84 | CH₃CH₂ | (CH₃)₂CH | pyrid-3-yl |
| 85 | CH₃CH₂ | (CH₃)₂CH | Ph-O |
| 86 | CH₃CH₂ | (CH₃)₂CH | 4-F-Ph-O |
| 87 | CH₃CH₂ | (CH₃)₂CH | 4-Cl-Ph-O |
| 88 | CH₃CH₂ | (CH₃)₂CH | pyrid-2-yl-O |
| 89 | CH₃CH₂ | cyclopropyl | Ph |
| 90 | CH₃CH₂ | cyclopropyl | 4-F-Ph |
| 91 | CH₃CH₂ | cyclopropyl | 4-Cl-Ph |
| 92 | CH₃CH₂ | cyclopropyl | pyrid-3-yl |
| 93 | CH₃CH₂ | cyclopropyl | Ph-O |
| 94 | CH₃CH₂ | cyclopropyl | 4-F-Ph-O |
| 95 | CH₃CH₂ | cyclopropyl | 4-Cl-Ph-O |
| 96 | CH₃CH₂ | cyclopropyl | Pyrid-2-yl-O |
| 97 | CH₃CH₂ | Ph | Ph |
| 98 | CH₃CH₂ | Ph | 4-F-Ph |
| 99 | CH₃CH₂ | Ph | 4-Cl-Ph |
| 100 | CH₃CH₂ | Ph | pyrid-3-yl |
| 101 | CH₃CH₂ | Ph | Ph-O |
| 102 | CH₃CH₂ | Ph | 4-F-Ph-O |
| 103 | CH₃CH₂ | Ph | 4-Cl-Ph-O |
| 104 | CH₃CH₂ | Ph | pyrid-2-yl-O |
| 105 | CH₃CH₂ | 4-F-Ph | Ph |
| 106 | CH₃CH₂ | 4-F-Ph | 4-F-Ph |
| 107 | CH₃CH₂ | 4-F-Ph | 4-Cl-Ph |
| 108 | CH₃CH₂ | 4-F-Ph | pyrid-3-yl |
| 109 | CH₃CH₂ | 4-F-Ph | Ph-O |
| 110 | CH₃CH₂ | 4-F-Ph | 4-F-Ph-O |
| 111 | CH₃CH₂ | 4-F-Ph | 4-Cl-Ph-O |
| 112 | CH₃CH₂ | 4-F-Ph | pyrid-2-yl-O |
| 113 | CH₃CH₂ | 4-Cl-Ph | Ph |
| 114 | CH₃CH₂ | 4-Cl-Ph | 4-F-Ph |
| 115 | CH₃CH₂ | 4-Cl-Ph | 4-Cl-Ph |
| 116 | CH₃CH₂ | 4-Cl-Ph | pyrid-3-yl |
| 117 | CH₃CH₂ | 4-Cl-Ph | Ph-O |
| 118 | CH₃CH₂ | 4-Cl-Ph | 4-F-Ph-O |
| 119 | CH₃CH₂ | 4-Cl-Ph | 4-Cl-Ph-O |
| 120 | CH₃CH₂ | 4-Cl-Ph | pyrid-2-yl-O |
| 121 | CH₃CH₂ | pyrid-3-yl | Ph |
| 122 | CH₃CH₂ | pyrid-3-yl | 4-F-Ph |
| 123 | CH₃CH₂ | pyrid-3-yl | 4-Cl-Ph |
| 124 | CH₃CH₂ | pyrid-3-yl | pyrid-3-yl |
| 125 | CH₃CH₂ | pyrid-3-yl | Ph-O |
| 126 | CH₃CH₂ | pyrid-3-yl | 4-F-Ph-O |
| 127 | CH₃CH₂ | pyrid-3-yl | 4-Cl-Ph-O |
| 128 | CH₃CH₂ | pyrid-3-yl | pyrid-2-yl-O |

where
a) 128 compounds of formula (I.a):

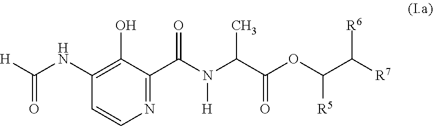

wherein R⁵, R⁶ and R⁷ are as defined in Table 1.
b) 128 compounds of formula (I.b):

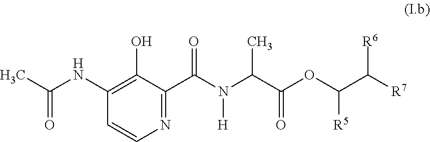

wherein R⁵, R⁶ and R⁷ are as defined in Table 1.
c) 128 compounds of formula (I.c):

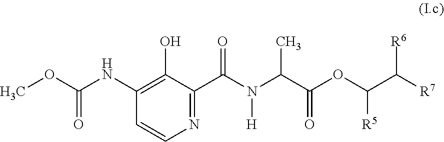

wherein R⁵, R⁶ and R⁷ are as defined in Table 1.
d) 128 compounds of formula (I.d):

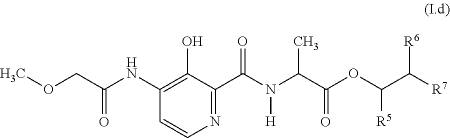

wherein R⁵, R⁶ and R⁷ are as defined in Table 1.
e) 128 compounds of formula (I.e):

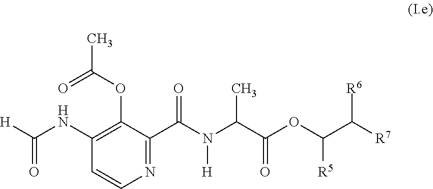

wherein R⁵, R⁶ and R⁷ are as defined in Table 1.
f) 128 compounds of formula (I.f):

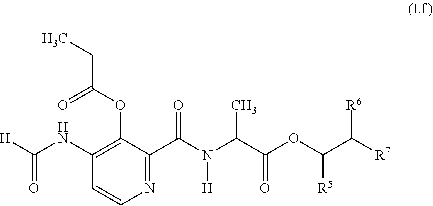

wherein R⁵, R⁶ and R⁷ are as defined in Table 1.
g) 128 compounds of formula (I.g):

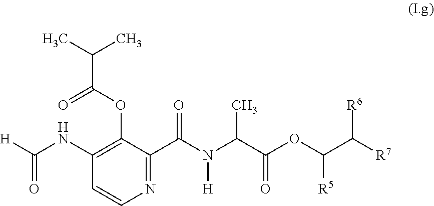

wherein R⁵, R⁶ and R⁷ are as defined in Table 1.
h) 128 compounds of formula (I.h):

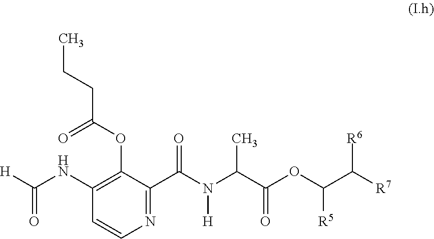

wherein R⁵, R⁶ and R⁷ are as defined in Table 1.
i) 128 compounds of formula (I.i):

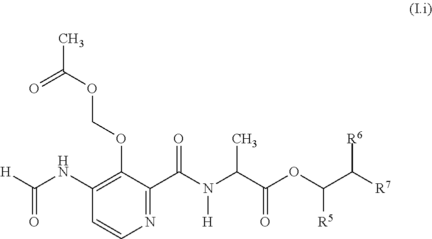

wherein R⁵, R⁶ and R⁷ are as defined in Table 1.
j) 128 compounds of formula (I.j):

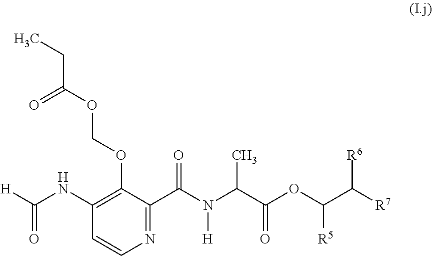

wherein R⁵, R⁶ and R⁷ are as defined in Table 1.
k) 128 compounds of formula (I.k):

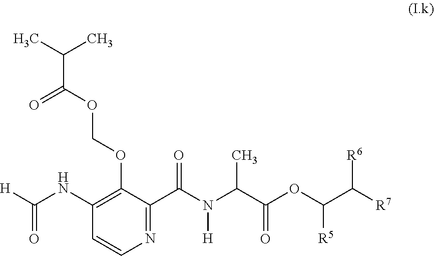

wherein R⁵, R⁶ and R⁷ are as defined in Table 1.
m) 128 compounds of formula (I.m):

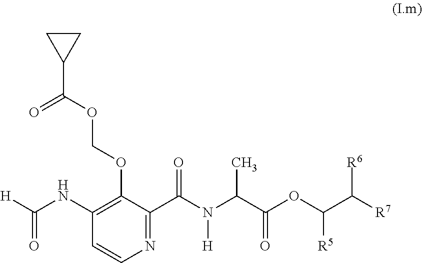

wherein R⁵, R⁶ and R⁷ are as defined in Table 1.
n) 128 compounds of formula (I.n):

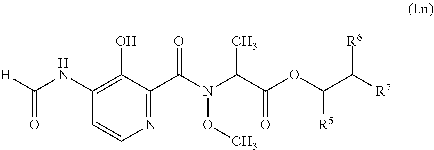

wherein R⁵, R⁶ and R⁷ are as defined in Table 1.
o) 128 compounds of formula (I.o):

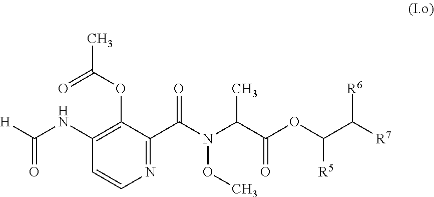

wherein R⁵, R⁶ and R⁷ are as defined in Table 1.
p) 128 compounds of formula (I.p):

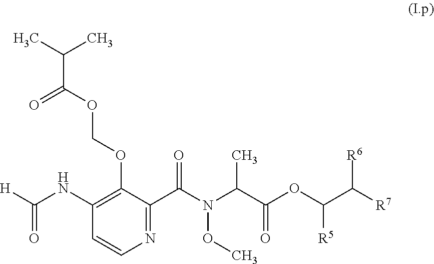

wherein R⁵, R⁶ and R⁷ are as defined in Table 1.
q) 128 compounds of formula (I.q):

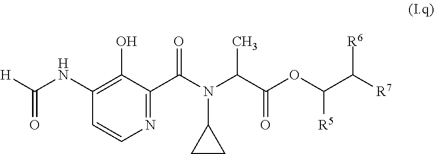

Wherein R⁵, R⁶ and R⁷ are as defined in Table 1.
r) 128 compounds of formula (I.r):

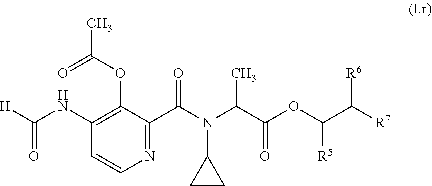

wherein R⁵, R⁶ and R⁷ are as defined in Table 1.
s) 128 compounds of formula (I.s):

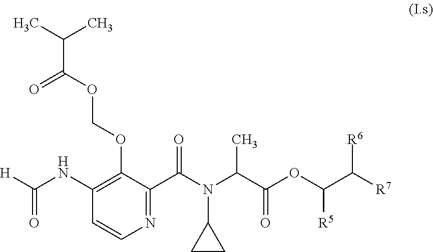

wherein R⁵, R⁶ and R⁷ are as defined in Table 1.
t) 128 compounds of formula (I.t):

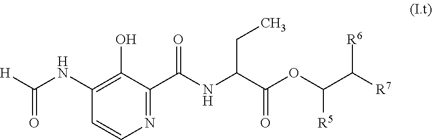

wherein R⁵, R⁶ and R⁷ are as defined in Table 1.
u) 128 compounds of formula (I.u):

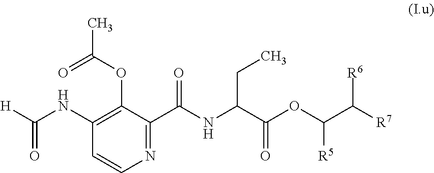

wherein R⁵, R⁶ and R⁷ are as defined in Table 1.
v) 128 compounds of formula (I.v):

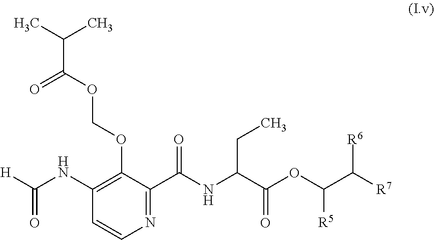

wherein R⁵, R⁶ and R⁷ are as defined in Table 1.
w) 128 compounds of formula (I.w):

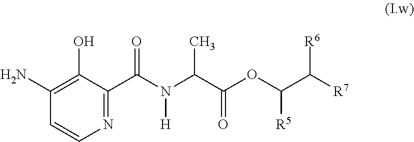

wherein R⁵, R⁶ and R⁷ are as defined in Table 1.
x) 128 compounds of formula (I.x):

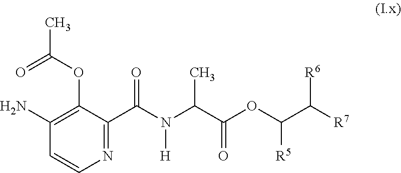

wherein R⁵, R⁶ and R⁷ are as defined in Table 1.
y) 128 compounds of formula (I.y):

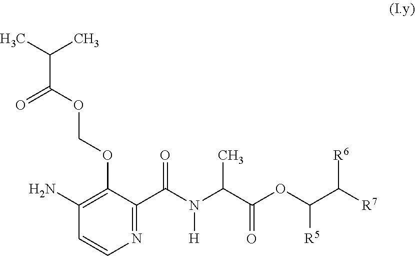

wherein R⁵, R⁶ and R⁷ are as defined in Table 1.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

Emulsifiable Concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

Extruder Granules

| | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

Coated Granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

### Suspension Concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### EXAMPLES

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

### LIST OF ABBREVIATIONS

° C.=degrees Celsius, CDCl₃=chloroform-d, EDC=1-ethyl-3-(3-dimethylaminopropyl)carbodiimide, HOBt=hydroxybenzotriazole, d=doublet, m=multiplet, MHz=mega hertz, mp=melting point, ppm=parts per million, s=singlet, t=triplet

### Example 1: This example illustrates the preparation of [2-[[(1S)-2-[2,2-bis(4-fluorophenyl)-1-methyl-ethoxy]-1-methyl-2-oxo-ethyl]carbamoyl]-4-formamido-3-pyridyl]oxymethyl 2-methylpropanoate (Compound I.k.42)

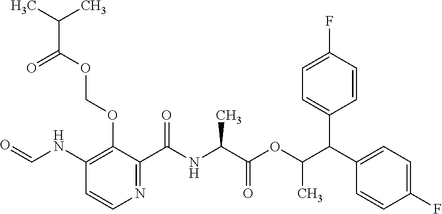 [SMILES: CC(C)C(=O)OCOc1c(NC=O)ccnc1C(=O)N[C@@H](C)C(=O)OC(C)C(c1ccc(F)cc1)c1ccc(F)cc1]

### a) Preparation of [2,2-bis(4-fluorophenyl)-1-methyl-ethyl] (2S)-2-[(4-azido-3-hydroxy-pyridine-2-carbonyl)amino]propanoate

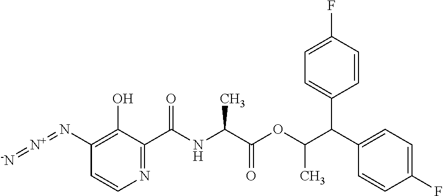

N-methylmorpholine (4.4 g, 43 mmol), HOBt (2.1 g, 15 mmol) and EDC (2.4 g, 15 mmol) were added successively to a solution of 4-azido-3-hydroxy-pyridine-2-carboxylic acid (2.1 g, 12 mmol) and [2,2-bis(4-fluorophenyl)-1-methyl-ethyl] (2S)-2-aminopropanoate (3.4 g, 11 mmol) in 53 ml of N,N-dimethylformamide. The reaction mixture was stirred for 16 h at room temperature, then diluted with ethyl acetate and water. After separation of the phases, the organic layer was washed with water, dried over sodium sulfate and concentrated under reduced pressure. The residue was purified by chromatography on silica gel, using ethyl acetate/heptane 1:3 as solvent system to deliver [2,2-bis(4-fluorophenyl)-1-methyl-ethyl] (2S)-2-[(4-azido-3-hydroxy-pyridine-2-carbonyl)amino]propanoate (2.1 g, 4.4 mmol). ¹H-NMR (400 MHz, CDCl₃): δ=1.08 (d, 3H), 1.27 (d, 3H), 4.06 (d, 1H), 4.49-4.55 (m, 1H), 5.70-5.76 (m, 1H), 6.95-7.33 (m, 8H), 7.98 (d, 1H), 8.30-8.36 (m, 1H), 12.39 (s, 1H).

### b) Preparation of [4-azido-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate

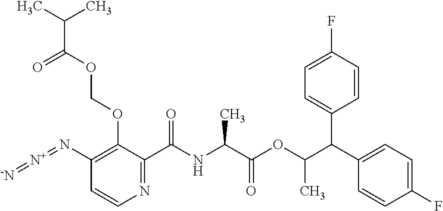

Sodium carbonate (0.13 g, 1.25 mmol), sodium iodide (62 mg, 0.4 mmol) and chloromethyl 2-methylpropanoate (0.11 g, 0.8 mmol) were added successively to a suspension of [2,2-bis(4-fluorophenyl)-1-methyl-ethyl] (2S)-2-[(4-azido-3-hydroxy-pyridine-2-carbonyl)amino]propanoate (0.2 g, 0.4 mmol) in 3 ml of acetone. The reaction mixture was stirred for 16 h at room temperature, then filtered. The filtrate was concentrated under reduced pressure, the residue was purified by chromatography on silica gel, using ethyl acetate/heptane 1:3 as solvent system to deliver [4-azido-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate (0.1 g, 0.2 mmol). ¹H-NMR (400 MHz, CDCl₃): δ=1.02 (d, 3H), 1.19-1.22 (m, 6H), 1.29 (d, 3H), 2.58-2.64 (m, 1H), 4.08 (d, 1H), 4.53-4.59 (m, 1H), 5.72-5.83 (m, 3H), 6.95-7.30 (m, 8H), 8.28 (d, 1H), 8.32-8.37 (m, 1H).

### c) Preparation of [4-amino-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate (Compound 1.y.42)

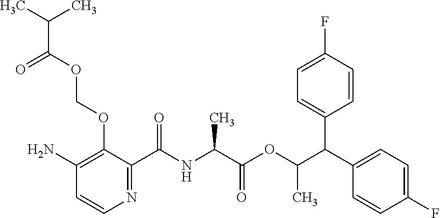 [SMILES: CC(C)C(=O)OCOc1c(N)ccnc1C(=O)N[C@@H](C)C(=O)OC(C)C(c1ccc(F)cc1)c1ccc(F)cc1]

Palladium on charcoal (0.1 g, 0.1 mmol) was added to a solution of [4-azido-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate (1.0 g, 1.7 mmol) in 8 ml of ethyl acetate and 4 ml of ethanol. The reaction mixture was stirred for 4 h at room temperature under a hydrogen atmosphere. Subsequently the reaction mixture was filtered through celite and concentrated under reduced pressure. The residue was purified by chromatography on silica gel, using ethyl acetate/heptane 1:3 as solvent system to deliver [4-amino-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate (Compound 1.y.42, 0.9 g, 1.6 mmol). ¹H-NMR (400 MHz, CDCl₃): δ=1.03 (d, 3H), 1.16-1.21 (m, 6H), 1.25 (d, 3H), 2.54-2.63 (m, 1H), 4.06 (d, 1H), 4.70-4.74 (m, 1H), 5.71-5.80 (m, 3H), 6.92-7.02 (m, 4H), 7.20-7.29 (m, 4H), 7.98 (d, 1H), 8.37-8.42 (m, 1H).

### d) Preparation of [2-[[(1S)-2-[2,2-bis(4-fluorophenyl)-1-methyl-ethoxy]-1-methyl-2-oxo-ethyl]carbamoyl]-4-formamido-3-pyridyl]oxymethyl 2-methylpropanoate (Compound 1.k.42)

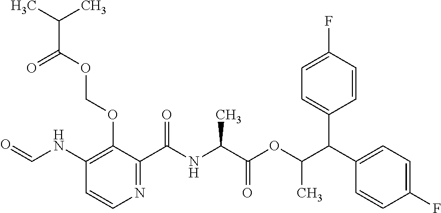 [SMILES: CC(C)C(=O)OCOc1c(NC=O)ccnc1C(=O)N[C@@H](C)C(=O)OC(C)C(c1ccc(F)cc1)c1ccc(F)cc1]

Formic acid (79 mg, 1.7 mmol) was added dropwise at 0° C. to acetic anhydride (0.14 g, 1.4 mmol). The resulting mixture was stirred for 15 min at 0° C. and for 15 min at room temperature, then heated for 16 h to 55° C. and cooled back to room temperature. This mixture was added to a solution of [4-amino-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate (Compound 1.y.42, 0.3 g, 0.54 mmol) in 9 ml of tetrahydrofuran.

The reaction mixture was stirred for 16 h at room temperature, then diluted with chloroform, filtered through celite and concentrated under reduced pressure. The residue was purified by chromatography on silica gel, using ethyl acetate/heptane 1:3 as solvent system to deliver [2-[[(1S)-2-[2,2-bis(4-fluorophenyl)-1-methyl-ethoxy]-1-methyl-2-oxo-ethyl]carbamoyl]-4-formamido-3-pyridyl]oxymethyl 2-methylpropanoate (Compound 1.k.42, 65 mg, 0.11 mmol). ¹H-NMR (400 MHz, CDCl₃): δ=1.05 (d, 3H), 1.16-1.21 (m, 6H), 1.27 (d, 3H), 2.59-2.64 (m, 1H), 4.07 (d, 1H), 4.55-4.59 (m, 1H), 5.73-5.82 (m, 3H), 6.92-7.02 (m, 4H), 7.20-7.29 (m, 4H), 8.32 (d, 1H), 8.54-8.59 (m, 1H), 8.93 (bs, 1H).

Throughout this description, temperatures are given in degrees Celsius (° C.) and “m.p.” means melting point. LC/MS means Liquid Chromatography Mass Spectrometry and the description of the apparatus and the method is: (ACQUITY UPLC from Waters, Phenomenex Gemini C18, 3 μm particle size, 110 Angström, 30×3 mm column, 1.7 mL/min., 60° C., H₂O+0.05% HCOOH (95%)/CH₃CN/MeOH 4:1+0.04% HCOOH (5%)—2 min.—CH₃CN/MeOH 4:1+0.04% HCOOH (5%)—0.8 min., ACQUITY SQD Mass Spectrometer from Waters, ionization method: electrospray (ESI), Polarity: positive ions, Capillary (kV) 3.00, Cone (V) 20.00, Extractor (V) 3.00, Source Temperature (° C.) 150, Desolvation Temperature (° C.) 400, Cone Gas Flow (L/Hr) 60, Desolvation Gas Flow (L/Hr) 700)).

TABLE 2
Melting point and LC/MS data for selected compounds of Table 1.

| | Compound | | | LC/MS |
|---|---|---|---|---|
| No. | Name | Structure | Mp (° C.) | Rt = Retention time |
| 1.a.42 | [2,2-bis(4- fluorophenyl)-1- methyl-ethyl] (2S)-2- [(4-formamido-3- hydroxy-pyridine-2- carbonyl)amino] propanoate | 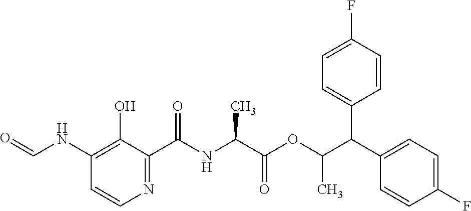 [SMILES: CC(OC(=O)[C@H](C)NC(=O)c1nccc(NC=O)c1O)C(c1ccc(F)cc1)c1ccc(F)cc1] | 101- 113 | |
| 1.k.42 | [2-[[(1S)-2-[2,2-bis(4- fluorophenyl)-1- methyl-ethoxy]-1- methyl-2-oxo- ethyl]carbamoyl]-4- formamido-3- pyridyl]oxymethyl 2- methylpropanoate | 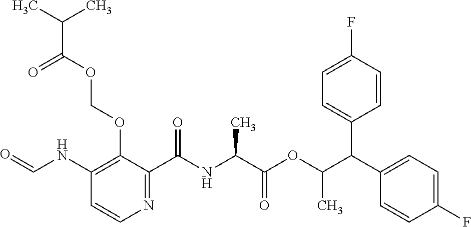 [SMILES: CC(C)C(=O)OCOc1c(NC=O)ccnc1C(=O)N[C@@H](C)C(=O)OC(C)C(c1ccc(F)cc1)c1ccc(F)cc1] | | Rt = 1.95 min; MS: m/z = 584 (M + 1) |
| 1.y.42 | [4-amino-2-[[(1S)-2- [2,2-bis(4- fluorophenyl)-1- methyl-ethoxy]-1- methyl-2-oxo- ethyl]carbamoyl]-3- pyridyl]oxymethyl 2- methylpropanoate | 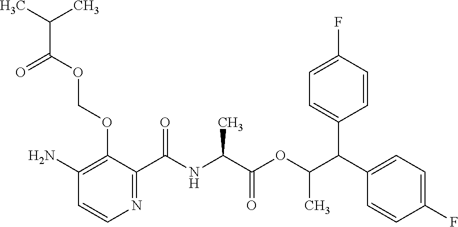 [SMILES: CC(C)C(=O)OCOc1c(N)ccnc1C(=O)N[C@@H](C)C(=O)OC(C)C(c1ccc(F)cc1)c1ccc(F)cc1] | | Rt = 1.48 min; MS: m/z = 557 (M + 1) |
| 1.e.42 | [2,2-bis(4- fluorophenyl)-1- methyl-ethyl] (2S)-2- [(3-acetoxy-4- formamido-pyridine-2- carbonyl)amino] propanoate | 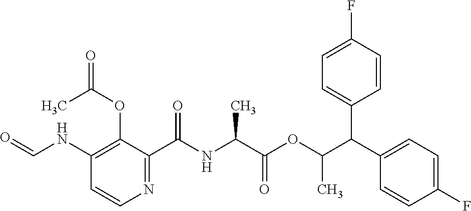 [SMILES: CC(=O)Oc1c(NC=O)ccnc1C(=O)N[C@@H](C)C(=O)OC(C)C(c1ccc(F)cc1)c1ccc(F)cc1] | | |
| 1.a.33 | (1-methyl-2,2- diphenyl-ethyl) (2S)- 2-[(4-formamido-3- hydroxy-pyridine-2- carbonyl)amino] propanoate | 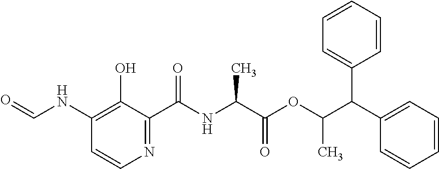 [SMILES: CC(OC(=O)[C@H](C)NC(=O)c1nccc(NC=O)c1O)C(c1ccccc1)c1ccccc1] | | |
| 1.k.33 | [4-formamido-2- [[(1S)-1-methyl-2-(1- methyl-2,2-diphenyl- ethoxy)-2-oxo- ethyl]carbamoyl]-3- pyridyl]oxymethyl 2- methylpropanoate | 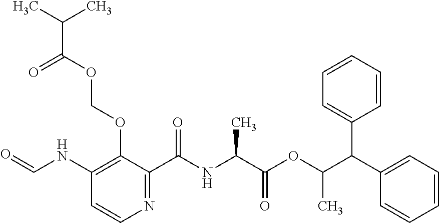 [SMILES: CC(C)C(=O)OCOc1c(NC=O)ccnc1C(=O)N[C@@H](C)C(=O)OC(C)C(c1ccccc1)c1ccccc1] | | Rt = 1.17 min; MS: m/z = 548 (M + 1) |
| 1.e.33 | (1-methyl-2,2- diphenyl-ethyl) (2S)- 2-[(3-acetoxy-4- formamido-pyridine-2- carbonyl)amino] propanoate | 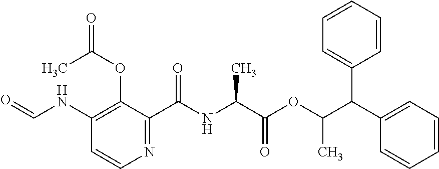 [SMILES: CC(=O)Oc1c(NC=O)ccnc1C(=O)N[C@@H](C)C(=O)OC(C)C(c1ccccc1)c1ccccc1] | | |
| 1.a.21 | (1,3-dimethyl-2- phenoxy-butyl) (2S)- 2-[(4-formamido-3- hydroxy-pyridine-2- carbonyl)amino] propanoate | 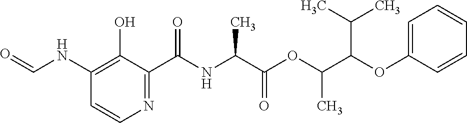 [SMILES: CC(C)C(Oc1ccccc1)C(C)OC(=O)[C@H](C)NC(=O)c1nccc(NC=O)c1O] | | |
| 1.k.21 | [2-[[(1S)-2-(1,3- dimethyl-2-phenoxy- butoxy)-1-methyl-2- oxo-ethyl]carbamoyl]- 4-formamido-3- pyridyl]oxymethyl 2- methylpropanoate | 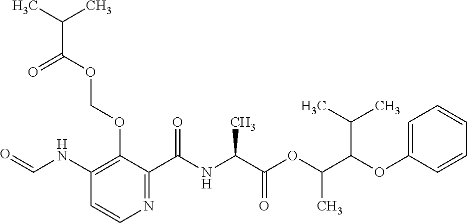 [SMILES: CC(C)C(=O)OCOc1c(NC=O)ccnc1C(=O)N[C@@H](C)C(=O)OC(C)C(Oc1ccccc1)C(C)C] | | |
| 1.e.21 | (1,3-dimethyl-2- phenoxy-butyl) (2S)- 2-[(3-acetoxy-4- formamido-pyridine-2- carbonyl)amino] propanoate | 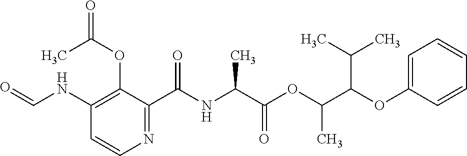 [SMILES: CC(=O)Oc1c(NC=O)ccnc1C(=O)N[C@@H](C)C(=O)OC(C)C(Oc1ccccc1)C(C)C] | | |
| 1.a.22 | [2-(4-fluorophenoxy)- 1,3-dimethyl-butyl] (2S)-2-[(4-formamido- 3-hydroxy-pyridine-2- carbonyl)amino] propanoate | 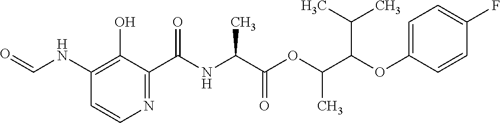 [SMILES: CC(C)C(Oc1ccc(F)cc1)C(C)OC(=O)[C@H](C)NC(=O)c1nccc(NC=O)c1O] | | |
| 1.k.22 | [2-[[(1S)-2-[2-(4- fluorophenoxy)-1,3- dimethyl-butoxy]-1- methyl-2-oxo- ethyl]carbamoyl]-4- formamido-3- pyridyl]oxymethyl 2- methylpropanoate | 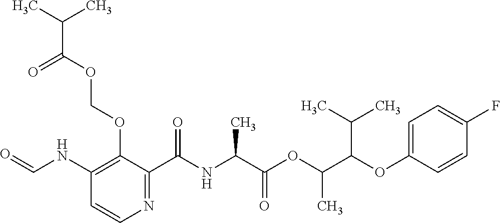 [SMILES: CC(C)C(=O)OCOc1c(NC=O)ccnc1C(=O)N[C@@H](C)C(=O)OC(C)C(Oc1ccc(F)cc1)C(C)C] | | |
| 1.e.22 | [2-(4-fluorophenoxy)- 1,3-dimethyl-butyl] (2S)-2-[(3-acetoxy-4- formamido-pyridine-2- carbonyl)amino] propanoate | 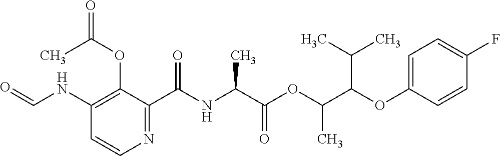 [SMILES: CC(=O)Oc1c(NC=O)ccnc1C(=O)N[C@@H](C)C(=O)OC(C)C(Oc1ccc(F)cc1)C(C)C] | | |
| 1.a.37 | (1-methyl-2-phenoxy- 2-phenyl-ethyl) (2S)- 2-[(4-formamido-3- hydroxy-pyridine-2- carbonyl)amino] propanoate | 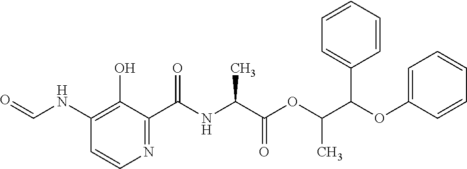 [SMILES: CC(OC(=O)[C@H](C)NC(=O)c1nccc(NC=O)c1O)C(Oc1ccccc1)c1ccccc1] | | |
| 1.k.37 | [4-formamido-2- [[(1S)-1-methyl-2-(1- methyl-2-phenoxy-2- phenyl-ethoxy)-2-oxo- ethyl]carbamoyl]-3- pyridyl]oxymethyl 2- methylpropanoate | 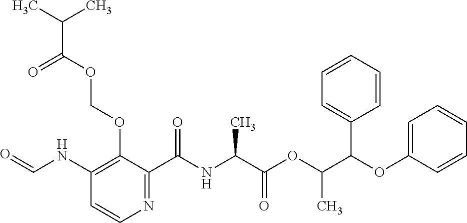 [SMILES: CC(C)C(=O)OCOc1c(NC=O)ccnc1C(=O)N[C@@H](C)C(=O)OC(C)C(Oc1ccccc1)c1ccccc1] | | |
| 1.e.37 | (1-methyl-2-phenoxy- 2-phenyl-ethyl) (2S)- 2-[(3-acetoxy-4- formamido-pyridine-2- carbonyl)amino] propanoate | 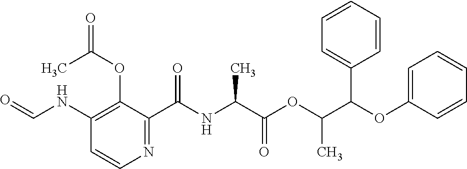 [SMILES: CC(=O)Oc1c(NC=O)ccnc1C(=O)N[C@@H](C)C(=O)OC(C)C(Oc1ccccc1)c1ccccc1] | | |
| 1.a.46 | [2-(4-fluorophenoxy)- 2-(4-fluorophenyl)-1- methyl-ethyl] (2S)-2- [(4-formamido-3- hydroxy-pyridine-2- carbonyl)amino] propanoate | 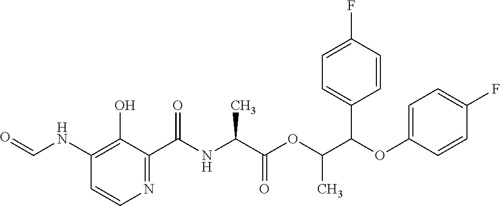 [SMILES: CC(OC(=O)[C@H](C)NC(=O)c1nccc(NC=O)c1O)C(Oc1ccc(F)cc1)c1ccc(F)cc1] | | |
| 1.k.46 | [2-[[(1S)-2-[2-(4- fluorophenoxy)-2-(4- fluorophenyl)-1- methyl-ethoxy]-1- methyl-2-oxo- ethyl]carbamoyl]-4- formamido-3- pyridyl]oxymethyl 2- methylpropanoate | 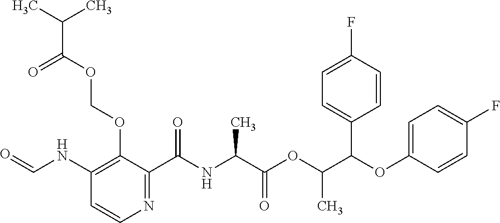 [SMILES: CC(C)C(=O)OCOc1c(NC=O)ccnc1C(=O)N[C@@H](C)C(=O)OC(C)C(Oc1ccc(F)cc1)c1ccc(F)cc1] | | |
| 1.e.46 | [2-(4-fluorophenoxy)- 2-(4-fluorophenyl)-1- methyl-ethyl] (2S)-2- [(3-acetoxy-4- formamido-pyridine-2- carbonyl)amino] propanoate | 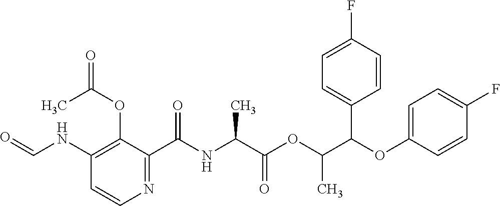 [SMILES: CC(=O)Oc1c(NC=O)ccnc1C(=O)N[C@@H](C)C(=O)OC(C)C(Oc1ccc(F)cc1)c1ccc(F)cc1] | | |
| 1.w.42 | [2,2-bis(4- fluorophenyl)-1- methyl-ethyl] (2S)-2- [(4-amino-3-hydroxy- pyridine-2-carbonyl) amino]propanoate | 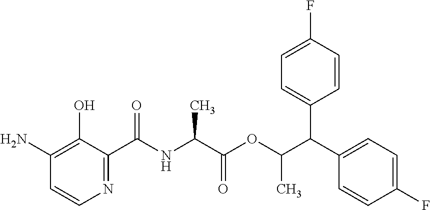 [SMILES: CC(OC(=O)[C@H](C)NC(=O)c1nccc(N)c1O)C(c1ccc(F)cc1)c1ccc(F)cc1] | | Rt = 1.44 min; MS: m/z = 457 (M + 1) |
| 1.y.33 | [4-amino-2-[[(1S)-1- methyl-2-(1-methyl- 2,2-diphenyl-ethoxy)- 2-oxo-ethyl] carbamoyl]-3-pyridyl] oxymethyl 2- methylpropanoate | 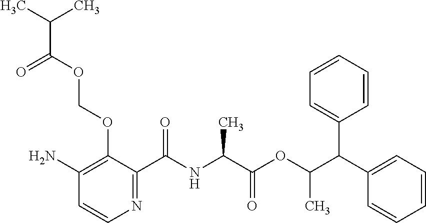 [SMILES: CC(C)C(=O)OCOc1c(N)ccnc1C(=O)N[C@@H](C)C(=O)OC(C)C(c1ccccc1)c1ccccc1] | | Rt = 0.94 min; MS: m/z = 521 (M + 1) |

### BIOLOGICAL EXAMPLES

Blumeria graminis f. sp. tritici (Erysiphe graminis f. sp. tritici)/Wheat/Leaf Disc Preventative (Powdery Mildew on Wheat)

Wheat leaf segments cv. Kanzler are placed on agar in a multiwell plate (24-well format) and sprayed with the test compound formulated with DMSO and Tween20 and diluted in water. The leaf disks are inoculated by shaking powdery mildew infected plants above the test plates 1 day after application. The inoculated leaf disks are incubated at 20° C. and 60% rh under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application). Compounds I.a.42, I.k.33, I.k.42 and I.y.42 at 200 ppm in the formulation give at least 80% disease control in this test when compared to untreated control leaf disks under the same conditions, which show extensive disease development.

Botryotinia fuckeliana (Botrytis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3-4 days after application. Compounds I.a.42, I.k.33, I.k.42 I.w.42, I.y.33 and I.y.42 at 200 ppm in the formulation give at least 80% disease control in this test when compared to untreated control leaf disks under the same conditions, which show extensive disease development.

Glomerella lagenarium (Colletotrichum lagenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3 to 4 days after application. Compound I.k.33, I.k.42 and I.w.42 at 200 ppm in the formulation give at least 80% disease control in this test when compared to untreated control leaf disks under the same conditions, which show extensive disease development.

Magnaporthe grisea (Pyricularia oryzae)/Rice/Leaf Disc Preventative (Rice Blast)

Rice leaf segments cv. Ballila are placed on agar in a multiwell plate (24-well format) and sprayed with the test compound formulated with DMSO and Tween20 and diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5 to 7 days after application). Compounds I.a.42, I.k.33, I.w.42, I.y.33 and I.y.42 at 200 ppm in the formulation gives at least 80% disease control in this test when compared to untreated control leaf disks under the same conditions, which show extensive disease development.

Monographella nivalis (Microdochium nivale)/liquid culture (foot rot cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application. Compounds I.a.42, I.k.33, I.k.42, I.w.42, I.y.33 and I.y.42 at 200 ppm in the formulation give at least 80% disease control in this test when compared to untreated control leaf disks under the same conditions, which show extensive disease development.

Mycosphaerella arachidis (Cercospora arachidicola)/Liquid Culture (Early Leaf Soot)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application. Compounds I.a.42, I.k.33, I.k.42, I.w.42, I.y.33 and I.y.42 at 200 ppm in the formulation give at least 80% disease control in this test when compared to untreated control leaf disks under the same conditions, which show extensive disease development.

Mycosphaerella graminicola (Septoria tritici)/Liquid Culture (Septoria Blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4 to 5 days after application. Compounds I.a.42, I.k.33, I.k.42, I.w.42, I.y.33 and I.y.42 at 200 ppm in the formulation give at least 80% disease control in this test when compared to untreated control leaf disks under the same conditions, which show extensive disease development.

Phaeosphaeria nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5 to 7 days after application). Compounds I.a.42, I.k.33, I.k.42, I.y.33 and I.y.42 at 200 ppm in the formulation give at least 80% disease control in this test when compared to untreated control leaf disks under the same conditions, which show extensive disease development.

Plasmopara viticola/Grape/Leaf Disc Preventative (Late Blight)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6 to 8 days after application). Compound I.a.42 at 200 ppm in the formulation give at least 80% disease control in this test when compared to untreated control leaf disks under the same conditions, which show extensive disease development.

Puccinia recondita f. sp. tritici/Wheat/Leaf Disc Preventative (Brown Rust)

Wheat leaf segments cv. Kanzler are placed on agar in multiwell plates (24-well format) and sprayed with the formulated test compound diluted in water. The leaf disks are inoculated with a spore suspension of the fungus 1 day after application. The inoculated leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (7 to 9 days after application). Compounds I.a.42, I.k.33, I.k.42, I.w.42, I.y.33 and I.y.42 at 200 ppm in the formulation give at least 80% disease control in this test when compared to untreated control leaf disks under the same conditions, which show extensive disease development.

Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application. Compound I.a.42 at 200 ppm in the formulation give at least 80% disease control in this test when compared to untreated control leaf disks under the same conditions, which show extensive disease development.

Phakopsora pachyrhizi/Soybean/Preventative (Soybean Rust)

Soybean leaf disks are placed on water agar in multiwell plates (24-well format) and sprayed with the formulated test compound diluted in water. One day after application leaf discs are inoculated by spraying a spore suspension on the lower leaf surface. After an incubation period in a climate cabinet of 24-36 hours in darkness at 20° C. and 75% rh leaf disc are kept at 20° C. with 12 h light/day and 75% rh. The activity of a compound is assessed as percent disease control compared to untreated when an appropriate level of disease damage appears in untreated check leaf disks (12-14 days after application).

1. A compound of formula (I): 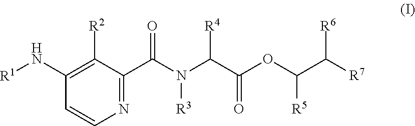
wherein,
R¹ is hydrogen, formyl, C₁-C₁₂alkylcarbonyl, C₁-C₆alkoxycarbonyl, C₁-C₆haloalkylcarbonyl, C₁-C₆alkoxyC₁-C₆alkylcarbonyl, or C₁-C₆haloalkoxycarbonyl;
R² is hydroxyl, C₂-C₆acyloxy, C₂-C₆haloacyloxy, C₁-C₆alkoxyC₁-C₆alkoxy, C₁-C₆haloalkoxyC₁-C₆alkoxy, C₁-C₆alkoxyC₁-C₆haloalkoxy, C₂-C₆acyloxyC₁-C₆alkoxy, C₂-C₆haloacyloxyC₁-C₆alkoxy, or C₂-C₆acyloxyC₁-C₆haloalkoxy;
R³ is hydrogen, C₁-C₆alkyl, C₁-C₆alkoxy or C₃-C₈cycloalkyl;
R⁴ and R⁵ are each independently C₁-C₁₂alkyl, C₃-C₈cycloalkyl, C₁-C₆haloalkyl, C₁-C₆alkoxyC₁-C₆alkyl, wherein each C₃-C₈cycloalkyl moiety is optionally substituted with 1, 2 or 3 halogen atoms which may be the same or different;
R⁶ is C₁-C₁₂alkyl, C₃-C₈cycloalkyl, phenyl or heteroaryl, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸; or
R⁵ and R⁶ together with the carbon atoms to which they are attached may form a 3-, 4-, 5- or 6-membered cycloalkyl or heterocycloalkyl ring, wherein the heterocyclic moiety is a stable 3-, 4-, 5- or 6-membered non-aromatic monocyclic ring which comprises 1, 2 or 3 heteroatoms, wherein the heteroatoms are individually selected from N, O and S;
R⁷ is phenyl, phenoxy, heteroaryl or heteroaryloxy, wherein the heteroaryl moiety is a 5- or 6-membered aromatic ring which comprises 1, 2, 3 or 4 heteroatoms individually selected from N, O and S, and wherein the phenyl and heteroaryl moieties are optionally substituted by 1, 2, 3 or 4 substituents, which may be the same or different, selected from R⁸;
R⁸ is hydroxyl, halogen, cyano, C₁-C₆alkyl, C₂-C₆alkenyl, C₂-C₆alkynyl, C₁-C₄haloalkyl, cyanoC₁-C₆alkyl, hydroxyC₁-C₆alkyl, or C₁-C₄alkoxyC₁-C₆alkyl;
or a salt or an N-oxide thereof.

2. The compound according to claim 1, wherein R¹ is hydrogen, formyl, C₁-C₆alkylcarbonyl, C₁-C₆alkoxycarbonyl, or C₁-C₆alkoxyC₁-C₆alkylcarbonyl.

3. The compound according to claim 1, wherein R² is hydroxyl, C₂-C₆acyloxy, C₁-C₆alkoxyC₁-C₆alkoxy, or C₂-C₆acyloxyC₁-C₆alkoxy.

4. The compound according to claim 1, wherein R³ is C₁-C₄alkyl, C₁-C₄alkoxy or C₃-C₆cycloalkyl.

5. The compound according to claim 1, wherein R³ is hydrogen.

6. The compound according to claim 1, wherein R⁴ and R⁵ are each independently C₁-C₄alkyl, C₃-C₆cycloalkyl or C₁-C₄haloalkyl.

7. The compound according to claim 1, wherein R⁴ and R⁵ are both C₁-C₄alkyl.

8. The compound according to claim 1, wherein R⁶ is C₁-C₆alkyl, C₃-C₆cycloalkyl, phenyl or heteroaryl, wherein the heteroaryl moiety is a 5- or 6-membered aromatic ring which comprises 1, 2 or 3 heteroatoms individually selected from N, O and S, and wherein the phenyl and heteroaryl moieties are optionally substituted by 1, 2 or 3 substituents, which may be the same or different, selected from R⁸.

9. The compound according to claim 1, wherein R⁷ is phenyl, phenoxy, heteroaryl or heteroaryloxy, wherein the heteroaryl moiety is a 5- or 6-membered aromatic ring which comprises 1, 2 or 3 heteroatoms individually selected from N, O and S, and wherein the phenyl and heteroaryl moieties are optionally substituted by 1, 2 or 3 substituents, which may be the same or different, selected from R⁸.

10. The compound according to claim 1, wherein R⁸ is hydroxyl, halogen, cyano, C₁₋₄alkyl, or C₁₋₄haloalkyl.

11. The compound according to claim 1 selected from:
[4-amino-2-[[(1S)-2-[2,2-bis(4-fluorophenyl)-1-methyl-ethoxy]-1-methyl-2-oxo-ethyl]carbamoyl]-3-pyridyl]oxymethyl 2-methylpropanoate;
[2,2-bis(4-fluorophenyl)-1-methyl-ethyl] (2S)-2-[(4-formamido-3-hydroxy-pyridine-2-carbonyl)amino]propanoate;
[2-[[(1S)-2-[2,2-bis(4-fluorophenyl)-1-methyl-ethoxy]-1-methyl-2-oxo-ethyl]carbamoyl]-4-formamido-3-pyridyl]oxymethyl 2-methylpropanoate;
[2,2-bis(4-fluorophenyl)-1-methyl-ethyl] (2S)-2-[(3-acetoxy-4-formamido-pyridine-2-carbonyl)amino]propanoate;
(1-methyl-2,2-diphenyl-ethyl) (2S)-2-[(4-formamido-3-hydroxy-pyridine-2-carbonyl)amino]propanoate;
[4-formamido-2-[[(1S)-1-methyl-2-(1-methyl-2,2-diphenyl-ethoxyl)-2-oxo-ethyl]carbamoyl]-3-pyridyl]oxymethyl 2-methylpropanoate;
(1-methyl-2,2-diphenyl-ethyl) (2S)-2-[(3-acetoxy-4-formamido-pyridine-2-carbonyl)amino]propanoate;
(1,3-dimethyl-2-phenoxy-butyl) (2S)-2-[(4-formamido-3-hydroxy-pyridine-2-carbonyl)amino]propanoate;
[2-[[(1S)-2-(1,3-dimethyl-2-phenoxy-butoxyl)-1-methyl-2-oxo-ethyl]carbamoyl]-4-formamido-3-pyridyl]oxymethyl 2-methylpropanoate;
(1,3-dimethyl-2-phenoxy-butyl) (2S)-2-[(3-acetoxy-4-formamido-pyridine-2-carbonyl)amino]propanoate;
[2-(4-fluorophenoxyl)-1,3-dimethyl-butyl] (2S)-2-[(4-formamido-3-hydroxy-pyridine-2-carbonyl)amino]propanoate;
[2-[[(1S)-2-[2-(4-fluorophenoxyl)-1,3-dimethyl-butoxy]-1-methyl-2-oxo-ethyl]carbamoyl]-4-formamido-3-pyridyl]oxymethyl 2-methylpropanoate;
[2-(4-fluorophenoxyl)-1,3-dimethyl-butyl] (2S)-2-[(3-acetoxy-4-formamido-pyridine-2-carbonyl)amino]propanoate;
(1-methyl-2-phenoxy-2-phenyl-ethyl) (2S)-2-[(4-formamido-3-hydroxy-pyridine-2-carbonyl)amino]propanoate;
[4-formamido-2-[[(1S)-1-methyl-2-(1-methyl-2-phenoxy-2-phenyl-ethoxyl)-2-oxo-ethyl]carbamoyl]-3-pyridyl]oxymethyl 2-methylpropanoate;
(1-methyl-2-phenoxy-2-phenyl-ethyl) (2S)-2-[(3-acetoxy-4-formamido-pyridine-2-carbonyl)amino]propanoate;
[2-(4-fluorophenoxyl)-2-(4-fluorophenyl)-1-methyl-ethyl] (2S)-2-[(4-formamido-3-hydroxy-pyridine-2-carbonyl)amino]propanoate;
[2-[[(1S)-2-[2-(4-fluorophenoxyl)-2-(4-fluorophenyl)-1-methyl-ethoxy]-1-methyl-2-oxo-ethyl]carbamoyl]-4-formamido-3-pyridyl]oxymethyl 2-methylpropanoate;
[2-(4-fluorophenoxyl)-2-(4-fluorophenyl)-1-methyl-ethyl] (2S)-2-[(3-acetoxy-4-formamido-pyridine-2-carbonyl)amino]propanoate;
[2,2-bis(4-fluorophenyl)-1-methyl-ethyl] (2S)-2-[(4-amino-3-hydroxy-pyridine-2-carbonyl) amino]propanoate; and
[4-amino-2-[[(1S)-1-methyl-2-(1-methyl-2,2-diphenyl-ethoxyl)-2-oxo-ethyl]carbamoyl]-3-pyridyl]oxymethyl 2-methylpropanoate.

12. An agrochemical composition comprising a fungicidally effective amount of a compound of formula (I) according to claim 1.

13. The composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound of formula (I) according to claim 1, or a composition comprising this compound as active ingredient, is applied to the plants, to parts thereof or the locus thereof.

15. Use of a compound of formula (I) according to claim 1 as a fungicide.
